(19) United States
(12) Patent Application Publication
(10) Pub. No.: US 2026/0209703 A1
(43) Pub. Date: Jul. 23, 2026

(54) DUAL CAR-T CELLS

(71) Applicant: CELLECTIS S.A., Paris (FR)

(72) Inventors: André CHOULIKA, Paris (FR); Laurent POIROT, Paris (FR); Beatriz ARANDA ORGILLES, Paris (FR); Philippe DUCHATEAU, Paris (FR)

(21) Appl. No.: 19/636,005

(22) Filed: Apr. 1, 2026

Related U.S. Application Data
(63) Continuation of application No. 18/016,972, filed on Jul. 30, 2021, now Pat. No. 12,618,048.

(30) Foreign Application Priority Data
Jul. 31, 2020 (DK) PA202070509

(51) Int. Cl. C12N 5/0783; A61K 40/11; A61K 40/31; A61K 40/42; A61P 35/00; C07K 16/28

(52) U.S. Cl. CPC C12N 5/0636; A61K 40/11; A61K 40/31; A61K 40/4212; A61K 40/4221; A61P 35/00; C07K 16/2887; C07K 16/2896; A61K 2239/29; A61K 2239/31; A61K 2239/38; A61K 2239/48; C07K 2317/53; C07K 2317/565; C07K 2319/03

(57) ABSTRACT

New engineered immune cells expressing two CARs directed against two different targets, polynucleotides for preparing said immune cells, pharmaceutical compositions comprising said immune cells, and the use of said immune cells in the treatment of cancers are provided herein.

### CROSS-REFERENCE TO RELATED APPLICATIONS

This application is a continuation of U.S. Ser. No. 18/016,972, filed on Jan. 19, 2023, which is a National Stage application under 35 U.S.C. § 371 of International Application No. PCT/EP2021/071400, having an International Filing Date of Jul. 30, 2021, which claims benefit of priority to DK Application Serial No. PA202070509, filed Jul. 31, 2020. The disclosures of the prior applications are considered part of (and are incorporated by reference in) the disclosure of this application.

### SEQUENCE LISTING

This application contains a Sequence Listing that has been submitted electronically as an XML file named “47246-0013002.XML.” The XML file, created on Mar. 27, 2026, is 62,324 bytes in size. The material in the XML file is hereby incorporated by reference in its entirety.

### FIELD

The present invention relates to the field of cell immunotherapy and more particularly to new engineered immune cells expressing two CARs directed against two different targets, useful in the treatment of cancers.

### BACKGROUND

Approximately every 3 minutes one person in the United States (US) is diagnosed with a blood cancer. An estimated combined total of 178,520 people in the US are expected to be diagnosed with leukemia, lymphoma or myeloma in 2020. New cases of leukemia, lymphoma and myeloma are expected to account for 9.9 percent of the estimated 1,806,590 new cancer cases diagnosed in the US in 2020 (Cancer Facts & Figures, 2020. American Cancer Society).

The development of chimeric antigen receptor (CAR) T-cell therapy for hematological malignancies represents one of the most remarkable therapeutic advances in the past decade (Holstein et al, 2020, Clin. Pharmacol. Ther. 107(1): 112-122). Indeed, as a rapidly progressing field in oncology, the adoptive transfer of CAR-T cells has shown striking efficacy in the management of hematological malignancies and has been reported in a number of clinical trials.

Chimeric antigen receptors (“CAR”) expressing immune cells are cells which have been genetically engineered to express CARs usually designed to recognize specific tumor antigens and kill the cancer cells that express these tumor antigens. It is not excluded that the CAR immune cells can activate the immune system to eliminate tumors. These are generally T cells expressing CARs (“CAR-T cells”) or Natural Killer cells expressing CARs (“CAR-NK cells”) or macrophages expressing CARs.

CARs are synthetic receptors consisting of a targeting moiety that is associated with one or more signalling domains in a single fusion molecule. In general, the binding moiety of a CAR consists of an antigen-binding domain derived from a monoclonal antibody, consisting of a single chain variable fragment (scFv), which contains the light and heavy variable fragments of a monoclonal antibody joined by a flexible linker. Binding moieties based on receptor or ligand domains have also been used successfully. The signalling domains for first generation CARs are derived from the cytoplasmic region of the CD3zeta or the Fc receptor gamma chains. First generation CARs have been shown to successfully redirect T cell cytotoxicity, however, they failed to provide prolonged expansion and anti-tumor activity in vivo. Signalling domains from co-stimulatory molecules including CD28, OX-40 (CD134), ICOS and 4-1BB (CD137) have been added alone (second generation) or in combination (third generation) to enhance survival and increase proliferation of CAR modified T cells. CARs have successfully allowed T cells to be redirected against antigens expressed at the surface of tumor cells from various malignancies including lymphomas and solid tumors (Jena, Dotti et al. 2010, Blood 116(7):1035-44).

Adoptive immunotherapy, which involves the transfer of autologous or allogeneic antigen-specific T cells generated ex vivo, is a promising strategy to treat viral infections and cancer as confirmed by the increase in the number of CAR-T cells approved by the US Food and Drug Administration (FDA) (e.g. Novartis' anti-CD19 CAR-T tisagenlecleucel (Kymriah™) for the treatment of precursor B-cell acute lymphoblastic leukemia, Kite Pharma's anti-CD19 CAR-T axicabtagene ciloleucel (Yescarta™) for certain types of large B-cell lymphoma in adult patients).

Despite the progress in research and development of CAR-T cell therapy, there still remains a need for improved CAR-T cells which could target a wider range of cancers as well as recurrent cancers and/or cancers for which the expression of the cancer-associated antigens are very variable and evolve with the time or during or after the treatments.

### SUMMARY

The inventors have developed new CAR-T cells targeting CD20 and CD22 antigens, which can be activated by the tumor cells expressing CD20 and CD22 at variable levels, and constitute an improvement over CAR-T cells of the prior art.

A first aspect relates to a genetically engineered immune cell expressing a Chimeric Antigen Receptor (CAR) specific for CD22 (CAR22) and a Chimeric Antigen Receptor specific for CD20 (CAR20) at its cell surface,
a) wherein said CAR22 comprises:
   i) at least one extracellular domain comprising:
      an antigen binding domain specific for CD22 comprising the Variable Heavy chain (VH) of SEQ ID NO: 11 and the Variable Light chain (VL) of SEQ ID NO: 12, optionally a leader sequence,
      a hinge domain from CD8alpha,
   ii) a transmembrane domain from CD8alpha, and
   iii an intracellular domain comprising a 4-1BB stimulatory domain and a CD3zeta signalling domain; and
b) wherein said CAR20 comprises:
   i) at least one extracellular domain comprising:
      an antigen binding domain specific for CD20 comprising the Variable Heavy chain (VH) of SEQ ID NO: 15 and the Variable Light chain (VL) of SEQ ID NO: 16, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain.

In a particular aspect, said genetically engineered immune cell is a TCR negative T-cell.

Another aspect concerns a pharmaceutical composition comprising said engineered immune cells or a population of cells comprising said engineered immune cells, and a pharmaceutically acceptable excipient.

A still other aspect relates to an isolated polynucleotide comprising:
a) a nucleic acid encoding a CAR22 comprising:
   i) at least one extracellular domain comprising:
      an antigen binding domain specific for CD22 comprising the Variable Heavy chain (VH) of SEQ ID NO: 11 and the Variable Light chain (VL) of SEQ ID NO: 12, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain; and
b) a nucleic acid encoding a CAR20 comprising:
   i) at least one extracellular domain comprising:
      an antigen binding domain specific for CD20 comprising the Variable Heavy chain (VH) of SEQ ID NO: 15 and the Variable Light chain (VL) of SEQ ID NO: 16, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain.

Other aspects concern a vector comprising said polynucleotides, as well as a host cell comprising said polynucleotides or vector.

A still further aspect relates to a method of preparing said engineered immune cells.

Other aspects concern said engineered immune cells or population of cells for use as a medicament.

Another aspect relies on said engineered immune cells for use in the treatment of a cancer or an inflammatory disorder, in particular a cancer or inflammatory disorder associated with CD20 and/or CD22 expression.

### BRIEF DESCRIPTION OF THE FIGURES

FIG. 1: CD20xCD22 or CD22xCD20 CAR detection. Flow cytometry analysis showing detection of CAR CD20 or CAR CD22 in non-transduced (NT), or T cells transduced with CD22 or CD22xCD20 or CD20xCD22 CAR constructs, using the protocol described in the Example.

FIG. 2: Flow cytometry analysis of the different Raji cells obtained after CD20 and CD22 TALEN® treatment and cell sorting.

FIG. 3: Percentage of lysis of CD20xCD22, CD22xCD20 and CD22 CAR T cells from 2 different donors against Raji cells expressing, or not, CD20 and/or CD22 antigens.

FIG. 4: Percentage of lysis, over time, of CD20xCD22, CD22xCD20 and CD22 CAR T cells against Raji cells either expressing CD20 and CD22 (A), expressing CD22 only (B), or expressing CD20 only (C).

FIG. 5: Dose dependent control of in vivo tumor burden upon treatment with indicated dose of CD20xCD22, CD22xCD20 and CD22 CAR T cells measured by bioluminescence.

FIG. 6: Survival curve for animals treated with the indicated dose of CD20xCD22, CD22xCD20 and CD22 CAR T-cells in a disseminated model of B-cell lymphoma.

FIG. 7: Survival curve of animals treated with the indicated doses of CD20xCD22, CD22xCD20 and CD22 CAR T-cells in a subcutaneous model of B-cell lymphoma.

FIG. 8. Release of IFN gamma by CD20xCD22, CD22xCD20, CD20 and CD22 CAR T cells on Daudi cells.

Corresponding nomenclatures used along the specification and in the examples are as follows:

$$Anti - CD 20 CAR = CAR 20 = CD 20 CAR$$

$$Anti - CD 22 CAR = CAR 22 = CD 22 CAR$$

$$Anti - CD 20 CAR / a nti - CD 22 CAR = CAR 20 \times 22 or$$

$$CAR 22 \times 20 = CD 20 \times CD 22 CAR or CD 22 \times CD 20 CAR$$

### DETAILED DESCRIPTION

Unless specifically defined herein, all technical and scientific terms used have the same meaning as commonly understood by a skilled artisan in the fields of gene therapy, biochemistry, genetics, and molecular biology.

All methods and materials similar or equivalent to those described herein can be used in the practice or testing of the present invention, with suitable methods and materials being described herein. All publications, patent applications, patents, and other references mentioned herein are incorporated by reference in their entirety. In case of conflict, the present specification, including definitions, will prevail. Further, the materials, methods, and examples are illustrative only and are not intended to be limiting, unless otherwise specified.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gait ed., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), specifically, Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); and Manipulating the Mouse Embryo, (Cold Spring Harbor Laboratory Press, Cold Spring Harbor, N.Y., 1986).

### Definitions

As used herewith “an antigen associated with a disease state” refers to an antigen present or over-expressed in a given disease. In the case of the antigen being CD20 or CD22, a “CD20-associated disease” or a “CD22-associated disease” refers to a disease like a cancer or an inflammatory disorder for which the CD20 or CD22 antigen is generally present on the tumor cells or the cells provoking the inflammatory reaction (B-cells in particular). An antigen associated with a disease state, wherein said disease state is a cancer, i.e. “an antigen associated with a cancer” can be a tumor antigen as defined herewith.

As used herein, the term “CD20” refers to an antigenic determinant known to be detectable on B-cells. Human CD20 is also called membrane-spanning 4-domains, subfamily A, member 1 (MS4A1). The human and murine amino acid and nucleic acid sequences can be found in a public database, such as GenBank, UniProt and Swiss-Prot. For example, the amino acid sequence of human CD20 can be found at Accession Nos. NP_690605.1 and NP_068769.2, and the nucleic acid sequence encoding transcript variants 1 and 3 of the human CD20 can be found at Accession No. NM_152866.2 and NM_021950.3, respectively. In one aspect, the antigen-binding portion of the CAR recognizes and binds an antigen within the extracellular domain of the CD20 protein. In one aspect, the CD20 protein is expressed on a cancer cell. As used herein, “CD20” includes proteins comprising mutations, e.g., point mutations, fragments, insertions, deletions and splice variants of full length wild-type CD20.

As used herein, the term “CD22” refers to an antigenic determinant known to be detectable on leukemia precursor cells. The human and murine amino acid and nucleic acid sequences can be found in a public database, such as GenBank, UniProt and Swiss-Prot. For example, the amino acid sequences of isoforms 1-5 human CD22 can be found at Accession Nos. NP 001762.2, NP 001172028.1, NP 001172029.1, NP 001172030.1, and NP 001265346.1, respectively, and the nucleic acid sequence encoding variants 1-5 of the human CD22 can be found at Accession No. NM 001771.3, NM 001185099.1, NM 001185100.1, NM 001185101.1, and NM 001278417.1, respectively. In one aspect, the antigen-binding portion of the CAR recognizes and binds an antigen within the extracellular domain of the CD22 protein. In one aspect, the CD22 protein is expressed on a cancer cell. As used herein, “CD22” includes proteins comprising mutations, e.g., point mutations, fragments, insertions, deletions and splice variants of full length wild-type CD22.

The term “tumor antigen” is meant to cover “tumor-specific antigen” and “tumor associated antigen”. Tumor-Specific Antigens (TSA) are generally present only on tumor cells and not on any other cell, while Tumor-Associated Antigens (TAA) are present on some tumor cells and also present on some normal cells. Tumor antigen, as meant herewith, also refers to mutated forms of a protein, which only appears in that form in tumors, while the non-mutated form is observed in non-tumoral tissues.

The term “extracellular antigen-binding domain” as used herein refers to an oligo- or poly-peptide that is capable of binding a specific antigen. Preferably, the domain will be capable of interacting with a cell surface molecule. For example, the extracellular antigen-binding domain may be chosen to recognize an antigen that acts as a cell surface marker on target cells associated with a particular disease state. In a particular instance, said extracellular antigen-binding domain comprises a single chain antibody fragment (scFv) comprising the light (V_{L}) and the heavy (V_{H}) variable fragment of a target-antigen-specific monoclonal antibody joined by a flexible linker. The antigen binding domain of a CAR expressed on the cell surface of the engineered immune cells described herewith can be any domain that binds to the target antigen and that derives from, for instance, a monoclonal antibody, a recombinant antibody, a human antibody, a humanized antibody, and a functional fragment thereof.

By “chimeric antigen receptor” or “CAR” is generally meant a synthetic receptor comprising a targeting moiety that is associated with one or more signalling domains in a single fusion molecule. As defined herewith, the term “chimeric antigen receptor” covers single chain CARs as well as multi-chain CARs. In general, the binding moiety of a CAR consists of an antigen-binding domain of a single-chain antibody (scFv), comprising the light and variable fragments of a monoclonal antibody joined by a flexible linker. Binding moieties based on receptor or ligand domains have also been used successfully. The signalling domains for first generation CARs are derived from the cytoplasmic region of the CD3zeta or the Fc receptor gamma chains. First generation CARs have been shown to successfully redirect T cell cytotoxicity. However, they failed to provide prolonged expansion and anti-tumor activity in vivo. Signalling domains from co-stimulatory molecules including CD28, OX-40 (CD134), and 4-1BB (CD137) have been added alone (second generation) or in combination (third generation) to enhance survival and increase proliferation of CAR modified T cells. CARs are not necessarily only single chain polypeptides, multi-chain CARs are also possible. According to the multi-chain CAR architecture, for instance as described in WO2014039523, the signalling domains and co-stimulatory domains are located on different polypeptide chains. Such multi-chain CARs can be derived from FcεRI, by replacing the high affinity IgE binding domain of FcεRI alpha chain by an extracellular ligand-binding domain such as scFv, whereas the N- and/or C-termini tails of FcεRI beta and/or gamma chains are fused to signal transducing domains and co-stimulatory domains respectively. The extracellular ligand binding domain has the role of redirecting T-cell specificity towards cell targets, while the signal transducing domains activate the immune cell response.

By “immune cell” is meant a cell of hematopoietic origin functionally involved in the initiation and/or execution of innate and/or adaptative immune response, such as typically CD45, CD3 or CD4 positive cells. The immune cell described herewith may be a dendritic cell, killer dendritic cell, a mast cell, macrophage, a natural killer cell (NK-cell), cytokine-induced killer cell (CIK cell), a B-cell or a T-cell selected from the group consisting of inflammatory T-lymphocytes, cytotoxic T-lymphocytes, regulatory T-lymphocytes or helper T-lymphocytes, gamma delta T cells, Natural killer T-cell (“NKT cell).

By “allogeneic” is meant that the cells originate from a donor, or are produced and/or differentiated from stem cells in view of being infused into patients having a different haplotype. Such immune cells are generally engineered to be less alloreactive and/or become more persistent with respect to their patient host. More specifically, the method of engineering allogeneic immune cells can comprise the step of reducing or inactivating TCR expression into T-cells, or into the stem cells to be derived into T-cells. This can be obtained by different sequence specific-reagents, such as by gene silencing or gene editing techniques (nuclease, base editing, shRNA, RNAi . . . ).

“Originating from a donor” means that the T-cells do not necessarily come directly from the donor as fresh cells, but may derive from stem cells or cell lines obtained from initial donors, who are not the treated patient (i.e. different haplotypes).

By “primary cell” or “primary cells” are intended cells taken directly from living tissue (e.g. biopsy material) and established for growth in vitro for a limited amount of time, meaning that they can undergo a limited number of population doublings. Primary cells are opposed to continuous tumorigenic or artificially immortalized cell lines. Non-limiting examples of such cell lines are CHO-K1 cells; HEK293 cells; Caco2 cells; U2-OS cells; NIH 3T3 cells; NSO cells; SP2 cells; CHO-S cells; DG44 cells; K-562 cells, U-937 cells; MRC5 cells; IMR90 cells; Jurkat cells; HepG2 cells; HeLa cells; HT-1080 cells; HCT-116 cells; Hu-h7 cells; Huvec cells; Molt 4 cells.

Primary immune cells can be obtained from a number of non-limiting sources, including peripheral blood mononuclear cells (PBMC), bone marrow, lymph node tissue, cord blood, thymus tissue, tissue from a site of infection, ascites, pleural effusion, spleen tissue, and from tumors, such as tumor infiltrating lymphocytes. In some embodiments, said immune cell can be derived from a healthy donor, from a patient diagnosed with cancer or from a patient diagnosed with an infection. In another embodiment, said cell is part of a mixed population of immune cells which present different phenotypic characteristics, such as comprising CD4, CD8 and CD56 positive cells. Primary immune cells are provided from donors or patients through a variety of methods known in the art, as for instance by leukapheresis techniques as reviewed by Schwartz J. et al. (Guidelines on the use of therapeutic apheresis in clinical practice-evidence-based approach from the Writing Committee of the American Society for Apheresis: the sixth special issue (2013) J Clin Apher. 28(3):145-284).

The immune cells derived from stem cells are also regarded as primary immune cells according to the present invention, in particular those deriving from induced pluripotent stem cells (iPS) [Yamanaka, K. et al. (2008). “Generation of Mouse Induced Pluripotent Stem Cells Without Viral Vectors”. Science. 322 (5903): 949-53]. Lentiviral expression of reprogramming factors has been used to induce multipotent cells from human peripheral blood cells [Staerk, J. et al. (2010). “Reprogramming of human peripheral blood cells to induced pluripotent stem cells”. Cell stem cell. 7 (1): 20-4][Loh, Y H. et al. (2010). “Reprogramming of T cells from human peripheral blood”. Cell stem cell. 7 (1): 15-9].

The immune cells may be derived from human embryonic stem cells by techniques well known in the art that do not involve the destruction of human embryos [Chung et al. (2008) Human Embryonic Stem Cell lines generated without embryo destruction, Cell Stem Cell 2(2):113-117].

By “Genetic engineering” is meant any methods aiming to introduce, modify and/or withdraw genetic material from a cell. By “gene editing” is meant a genetic engineering allowing genetic material to be added, removed, or altered at specific locations (loci) in the genome, including punctual mutations. Gene editing generally involves sequence specific reagents.

By “identity”, it is referred to sequence identity between two nucleic acid molecules or polypeptides. Identity can be determined by comparing a position in each sequence which may be aligned for purposes of comparison. When a position in the compared sequence is occupied by the same base, then the molecules are identical at that position. A degree of similarity or identity between nucleic acid or amino acid sequences is a function of the number of identical or matching nucleotides at positions shared by the nucleic acid sequences. Various alignment algorithms and/or programs may be used to calculate the identity between two sequences, including FASTA, or BLAST which are available as a part of the GCG sequence analysis package (University of Wisconsin, Madison, Wis.), and can be used with, e.g., default setting. For example, polypeptides having at least 80%, at least 85%, at least 90%, at least 95%, at least 98% or at least 99% identity to specific polypeptides described herein and preferably exhibiting substantially the same functions, as well as polynucleotide encoding such polypeptides, are contemplated. Unless otherwise stated, the present invention encompasses polypeptides and polynucleotides that have the same function and share at least 80%, generally at least 85%, preferably at least 90%, more preferably at least 95% and even more preferably at least 97% with those described herein.

The terms “patient” or “subject” and “donor” herein include all members of the animal kingdom including non-human primates and humans.

The invention is based on the surprising observation according to which a tumor cell expressing low levels of CD20 antigen and low levels of CD22 antigen, while not able to efficiently activate a CAR-T cell targeting either CD20 or CD22, could surprisingly activate a dual CAR-T cell expressing both a CAR targeting CD20 and a CAR targeting CD22.

Without willing to be bound by this theory, in these conditions, the total number of CD20 antigen molecules and CD22 antigen molecules per tumor site represents a threshold over which the dual CAR-T cell is activated, while a single CAR-T cell is not.

This could be represented as follows:
X=density of CD20 antigen molecules at the surface of the tumor, that are recognized by an anti-CD20 CAR (CAR20);
Y=density of CD22 antigen molecules at the surface of the tumor, that are recognized by an anti-CD22 CAR (CAR22);
X+Y=Z=density of CD20 antigen molecules and CD22 antigen molecules at the surface of the tumor;
T=density of antigen molecules bound to the T-cell that triggers said T-cell activation=threshold necessary for T-cell activation
If Z>T, then T-cell activation occurs.

TABLE 1
Schematic representation of the relation between CAR T-cell activation
and level of expression of the target antigens CD20 and CD22

| | TUMOR | | | | | | | |
|---|---|---|---|---|---|---|---|---|
| | | | | | CD20^{HIGH} | CD20^{low} | CD20^{HIGH} | CD20^{low} |
| CAR-T cell | CD20^{HIGH} | CD22^{HIGH} | CD20^{low} | CD22^{low} | CD22^{HIGH} | CD22^{HIGH} | CD22^{low} | CD22^{low} |
| CAR20-T | X > T | X < T | X < T | X < T | X > T | X < T | X > T | X < T |
| cell | T-cell | No T-cell | No T-cell | No T-cell | T-cell | No T-cell | T-cell | No T-cell |
| | activation | activation | activation | activation | activation | activation | activation | activation |
| CAR22-T | Y < T | Y > T | Y < T | Y < T | Y > T | Y > T | Y < T | Y < T |
| cell | No T-cell | T-cell | No T-cell | No T-cell | T-cell | T-cell | No T-cell | No T-cell |
| | activation | activation | activation | activation | activation | activation | activation | activation |
| CAR20x22- | Z > T | Z > T | Z < T | Z < T | Z > T | Z > T | Z > T | Z > T |
| T cell | T-cell | T-cell | No T-cell | No T-cell | T-cell | T-cell | T-cell | T-cell |
| | activation | activation | activation | activation | activation | activation | activation | activation |

One of the advantages of the T cells expressing both CAR20 and CAR22 (dual “CAR20x22-T cell” or “CAR22x20-T cell”) of the invention is, thus, to be useful in immunotherapy to target an extended population of tumor cells having different levels of expression of the CD20 and/or CD22 antigens. Thus, not only the tumor cells having a low level of expression of the CD20 antigen and a high level of expression of the CD22 antigen, as well as those having a high level of expression of CD20 antigen and a low level of expression of the CD22 antigen would be targeted (and thus killed) by the dual CAR22x20-T cells of the invention but, surprisingly, also the cells having low levels of expression of the CD20 and the CD22 antigens would be targeted and, thus, killed.

Another advantage of the dual CAR-T cells of the invention relies on their utility in immunotherapy to treat a tumor that evolves by expressing more or less of the CD20 and CD22 antigens along time or during the treatment.

Another advantage of the dual CAR-T cells of the invention relies on their utility in immunotherapy to treat cancers characterized by a low expression of CD20 and CD22 antigens.

A still other advantage of the dual CAR-T cells of the invention is related to the synergistic effect of the two CARs, possibly by favouring and strengthening the immune synapse between the T-cells and their target tumor cells, possibly allowing a higher level of effector cytokines production.

On top of the above-mentioned advantages, the dual CAR-T cells of the invention are also useful to avoid CD20- and CD22-associated cancer relapse and/or antigen escape.

What has been observed and described herewith is likely to be generalizable so that dual CAR-T cells targeting two different tumor associated antigens could thus be useful in immunotherapy to target an extended population of tumor cells having different levels of expression of the tumor associated antigens as detailed above.

A further surprising effect of one aspect of the invention is related to the observation made by the inventors according to which an immune cell expressing a CAR20 and a CAR22 at its cell surface and having, integrated in the cell's genome, an exogenous nucleic acid comprising, from 5′ to 3′:
(i) a promoter,
(ii) a nucleic acid encoding said CAR20,
(iii) a nucleic acid encoding a self-cleaving peptide;
(iv) a nucleic acid encoding said CAR22,
whereby the same promoter controls the expression of said CAR20 and CAR22;
exhibits a higher tumor reduction in vivo compared to an immune cell expressing a CAR20 and a CAR22 at its cell surface and having, integrated in the cell's genome, an exogenous nucleic acid comprising, from 5′ to 3′:
(i) a promoter,
(ii) a nucleic acid encoding said CAR22,
(iii) a nucleic acid encoding a self-cleaving peptide;
(iv) a nucleic acid encoding said CAR20.
   Immune cells Expressing anti-CD20 CAR and Anti-CD22 CAR

The immune cells described herewith are endowed with two synthetic chimeric antigen receptors (CARs) targeting the CD20 antigen and the CD22 antigen, respectively.

Anti-CD22 CAR (CAR22) and anti-CD20 CAR (CAR20)

The immune cells described herewith are endowed with two synthetic CARs, which confers them a higher specificity toward a cell, such as a tumor comprising cells expressing the CD20 and/or CD22 antigen or toward inflammatory cells expressing the CD20 and/or CD22 antigen.

A recombinant chimeric antigen receptor is generally encoded by an exogenous polynucleotide which is introduced into the cell using viral vectors as per one of the transduction steps referred to elsewhere in the current application. A recombinant receptor encoded by an exogenous polynucleotide can also be introduced into the cell in the form of a plasmid or a PCR product.

In general, CAR polypeptides comprise an extracellular antigen-binding domain, a transmembrane domain, and an intracellular domain comprising a costimulatory domain and/or a primary signalling domain, wherein said antigen binding domain binds to the antigen associated with the disease state.

While the anti-CD20 and anti-CD22 CARs described herewith are not limited to a specific CAR structure, a nucleic acid that can be used to genetically engineer the immune cells generally encodes a CAR comprising: an extracellular antigen-binding domain that binds to an antigen associated with a disease state, a hinge, a transmembrane domain, and an intracellular domain comprising a stimulatory domain and/or a primary signalling domain. Generally, the extracellular antigen-binding domain is a scFv comprising a Heavy variable chain (VH) and a Light variable chain (VL) of an antibody binding to a specific antigen (e.g., to a tumor antigen) connected via a Linker. The transmembrane domain can be, for example, a CD8a transmembrane domain or a 4-1BB transmembrane domain. The stimulatory domain can be, for example, the 4-1 BB stimulatory domain. The primary signalling domain can be, for example, the CD3ζ signalling domain.

In one embodiment, to avoid any recombination event within the construct comprising polynucleotides encoding two CARs comprising identical domains, the nucleotide acid sequences used to code for the same amino acid sequences present twice in the construct (e.g. the same transmembrane domain, the same stimulatory domain) are optimized using codon usage and code degeneracy so that the nucleotide sequences diverge.

TABLE 2
Sequence of different domains typically present in a CAR

| Functional | | |
|---|---|---|
| domains | SEQ ID # | amino acid sequence |
| CD8α signal | SEQ ID NO: 1 | MALPVTALLLPLALLLHAARP |
| peptide (or | | |
| sequence leader) | | |
| Alternative | SEQ ID NO: 2 | METDTLLLWVLLLWVPGSTG |
| signal | | |
| peptide | | |
| FcγRIIIα hinge | SEQ ID NO: 3 | GLAVSTISSFFPPGYQ |
| CD8α hinge | SEQ ID NO: 4 | TTTPAPRPPTPAPTIASQPLSLRPEACRPAAGGAV |
| | | HTRGLDFACD |
| IgG1 hinge | SEQ ID NO: 5 | EPKSPDKTHTCPPCPAPPVAGPSVFLFPPKPKDTL |
| | | MIARTPEVTCVVVDVSHEDPEVKFNWYVDGVEVHN |
| | | AKTKPREEQYNSTYRVVSVLTVLHQDWLNGKEYKC |
| | | KVSNKALPAPIEKTISKAKGQPREPQVYTLPPSRD |
| | | ELTKNQVSLTCLVKGFYPSDIAVEWESNGQPENNY |
| | | KTTPPVLDSDGSFFLYSKLTVDKSRWQQGNVFSCS |
| | | VMHEALHNHYTQKSLSLSPGK |
| CD8α | SEQ ID NO: 6 | IYIWAPLAGTCGVLLLSLVITLYC |
| transmembrane | | |
| domain | | |
| 4-1BB | SEQ ID NO: 7 | IISFFLALTSTALLFLLFFLTLRFSVV |
| transmembrane | | |
| domain | | |
| 4-1BB stimulatory | SEQ ID NO: 8 | KRGRKKLLYIFKQPFMRPVQTTQEEDGCSCRFPEE |
| domain | | EEGGCEL |
| CD3ζ signalling | SEQ ID NO: 9 | RVKFSRSADAPAYQQGQNQLYNELNLGRREEYDVL |
| domain | | DKRRGRDPEMGGKPRRKNPQEGLYNELQKDKMAEA |
| | | YSEIGMKGERRRGKGHDGLYQGLSTATKDTYDALH |
| | | MQALPPR |
| Linker | SEQ ID NO: 10 | GGGGSGGGGSGGGGS |

In one aspect, the antigen binding domain specific for CD20 comprises a Variable Heavy chain (VH) and a Variable Light chain (VL) linked by a linker, wherein said VH chain comprises the H-CDRs of SEQ ID NO: 47, SEQ ID NO: 48, and SEQ ID NO: 49, and said VL chain comprises the L-CDRs of SEQ ID NO: 50, SEQ ID NO: 51, and SEQ ID NO: 52.

For instance, the antigen binding domain specific for CD20 comprises a Variable Heavy chain (VH) and a Variable Light chain (VL) linked by a linker (forming the scFv of SEQ ID NO: 17), wherein said VH and VL chains comprise the H-CDRs of SEQ ID NO: 47, SEQ ID NO: 48, and SEQ ID NO: 49, and the L-CDRs of SEQ ID NO: 50, SEQ ID NO: 51, and SEQ ID NO: 52.

An anti-CD20 CAR as described herewith can comprise:
i) at least one extracellular domain comprising:
   an antigen binding domain specific for CD20 comprising a Variable Heavy chain (VH) comprising an amino acid sequence having at least 80% identity, at least 90%, at least 95%, at least 97%, at least 99%, or 100% identity with SEQ ID NO: 15 and comprising the H-CDRs of amino acid sequences SEQ ID NO: 47, SEQ ID NO: 48, and SEQ ID NO: 49, and a Variable Light chain (VL) comprising an amino acid sequence having at least 80%, at least 90%, at least 95%, at least 97%, at least 99%, or 100% identity with SEQ ID NO: 16 and comprising the L-CDRs of amino acid sequences SEQ ID NO: 50, SEQ ID NO: 51, and SEQ ID NO: 52, optionally a leader sequence,
   a hinge domain from CD8alpha,
ii) a transmembrane domain from CD8alpha, and
iii) an intracellular domain comprising a 4-1BB stimulatory domain and a CD3zeta signalling domain.

In a particular aspect, the CAR targeting the CD20 antigen present on tumor cells, which is expressed by the genetically engineered immune cells described herewith, is as described in Tables 3 and 4.

TABLE 3
Sequence of the VH and VL comprised
in the scFv of an anti-CD20 CAR
described herewith and illustrated in
the Example section

| | SEQ ID | |
|---|---|---|
| Region | # | amino acid sequence |
| Heavy | SEQ ID | EVQLVESGGGLVQPGRSLRLSCAASGF |
| variable | NO: 15 | TFNDYAMHWVRQAPGKGLEWVSTISWN |
| region | | SGSIGYADSVKGRFTISRDNAKKSLYL |
| | | QMNSLRAEDTALYYCAKDIQYGNYYYG |
| | | MDVWGQGTTVTVSS |
| Light | SEQ ID | EIVLTQSPATLSLSPGERATLSCRASQ |
| variable | NO: 16 | SVSSYLAWYQQKPGQAPRLLIYDASNR |
| region | | ATGIPARFSGSGSGTDFTLTISSLEPE |
| | | DFAVYYCQQRSNWPITFGQGTRLEIK |

TABLE 4
Structure of an anti-CD20 CAR described herewith
and illustrated in the Example section

| CAR | CAR Structure | | | | | | |
|---|---|---|---|---|---|---|---|
| Designation | signal | | | CD8α | CD8α | 4-1BB | CD3ζ |
| CAR20 | peptide | VH | VL | hinge | TM | IC | CD |
| (SEQ ID NO: | SEQ ID | SEQ ID | SEQ ID | SEQ ID | SEQ ID | SEQ ID | SEQ ID |
| 18) | NO: 1 | NO: 15 | NO: 16 | NO: 4 | NO: 6 | NO: 8 | NO: 9 |

In one aspect, the antigen binding domain specific for CD22 comprises a Variable Heavy chain (VH) and a Variable Light chain (VL) linked by a linker, wherein said VH chain comprises the H-CDRs of SEQ ID NO: 41, SEQ ID NO: 42, and SEQ ID NO: 43, and said VL chain comprises the L-CDRs of SEQ ID NO: 44, SEQ ID NO: 45, and SEQ ID NO: 46.

For instance, the antigen binding domain specific for CD22 comprises a Variable Heavy chain (VH) and a Variable Light chain (VL) linked by a linker (forming the scFv of SEQ ID NO: 13), wherein said VH and VL chains comprise the H-CDRs of SEQ ID NO: 41, SEQ ID NO: 42, and SEQ ID NO: 43, and the L-CDRs of SEQ ID NO: 44, SEQ ID NO: 45, and SEQ ID NO: 46.

An anti-CD22 CAR as described herewith can comprise:
i) at least one extracellular domain comprising:
   an antigen binding domain specific for CD22 comprising a Variable Heavy chain (VH) comprising an amino acid sequence having at least 80% identity, at least 90%, at least 95%, at least 97%, at least 99%, or 100% identity, with SEQ ID NO: 11 and comprising the H-CDRs of amino acid sequences SEQ ID NO: 41, SEQ ID NO: 42, and SEQ ID NO: 43, and a Variable Light chain (VL) comprising an amino acid sequence having at least 80%, at least 90%, at least 95%, at least 97%, at least 99%, or 100% identity, identity with SEQ ID NO: 12 and comprising the L-CDRs of amino acid sequences SEQ ID NO: 44, SEQ ID NO: 45, and SEQ ID NO: 46, optionally a leader sequence,
   a hinge domain from CD8alpha,
ii) a transmembrane domain from CD8alpha, and
iii an intracellular domain comprising a 4-1BB stimulatory domain and a CD3zeta signalling domain.

In a particular aspect, the CAR targeting the CD22 antigen present on tumor cells, which is expressed by the genetically engineered immune cells described herewith, is described in Tables 5 and 6 below and in the Example section.

TABLE 5
Sequence of the VH and VL comprised
in the scFv of an anti-CD22 CAR
described herewith and illustrated in
the Example section

| | SEQ ID | |
|---|---|---|
| Region | # | amino acid sequence |
| Heavy | SEQ ID | QVQLQQSGPGLVKPSQTLSLTCAISG |
| variable | NO: 11 | DSVSSNSAAWNWIRQSPSRGLEWLGR |
| region | | TYYRSKWYNDYAVSVKSRITINPDTS |
| | | KNQFSLQLNSVTPEDTAVYYCAREVT |
| | | GDLEDAFDIWGQGTMVTVSS |
| Light | SEQ ID | DIQMTQSPSSLSASVGDRVTITCRAS |
| variable | NO: 12 | QTIWSYLNWYQQRPGKAPNLLIYAAS |
| region | | SLQSGVPSRFSGRGSGTDFTLTISSL |
| | | QAEDFATYYCQQSYSIPQTFGQGTKL |
| | | EIK |

TABLE 6
Structure of an anti-CD22 CAR described herewith
and illustrated in the Example section

| CAR | CAR Structure | | | | | | |
|---|---|---|---|---|---|---|---|
| Designation | signal | | | CD8α | CD8α | 4-1BB | CD3ζ |
| CAR22 | peptide | VH | VL | hinge | TM | IC | CD |
| (SEQ ID NO: | SEQ ID | SEQ ID | SEQ ID | SEQ ID | SEQ ID | SEQ ID | SEQ ID |
| 14) | NO: 1 | NO: 11 | NO: 12 | NO: 4 | NO: 6 | NO: 8 | NO: 9 |

### Immune Cells Expressing CAR20 and CAR22

The engineered immune cells described herewith are endowed with the two synthetic chimeric antigen receptors (CARs) targeting the CD20 antigen and the CD22 antigen, respectively, as described herewith.

In a particular instance, said immune cells do not express any other CAR targeting another antigen than CD20 or CD22. More particularly, said immune cells do not express other CARs than the CAR22 and CAR20 described herewith.

The immune cell can be, for instance, a dendritic cell, killer dendritic cell, a mast cell, a macrophage, a NK-cell, a cytokine-induced Killer (CIK) cell, a B-cell or a T-cell selected from the group consisting of inflammatory T-lymphocytes, cytotoxic T-lymphocytes, regulatory T-lymphocytes or helper T-lymphocytes, gamma delta T cells, NKT cells and tumor infiltrating lymphocytes (TILL).

In a particular instance, the immune cell engineered to express the two CARs is selected from the group consisting of a T-cell, a NK-cell, and a macrophage.

In a more particular instance, the immune cell expressing the two CARs is a T-cell, for instance a cytotoxic T cell.

In a general instance, said immune cell is comprised in a population of cells, such as a population of immune cells, in particular a population of T-cells, a population of NK-cells, and/or a population of macrophages.

In a particular instance, said immune cell is a T-cell for use in off-the shelf immunotherapy.

In a particular instance, said engineered immune cell is a T-cell that is TCR negative (does not express TCR alpha at its cell surface).

In a particular instance said engineered T-cell expresses a short hairpin RNA (shRNA) or small interfering (siRNA) directed against a polynucleotide sequence encoding a component of TCR.

In another particular instance, said engineered T-cell is mutated in its TCR alpha and/or TCR beta alleles.

In particular, said engineered T-cell can have at least one allele encoding TCR alpha, TCR beta, and/or CD3 that has been inactivated by mutation.

In a still further instance, said engineered T-cell has at least one allele selected from β2m, PD1, CTLA4, dCK, CD52 and/or GR that has been inactivated.

In a still further instance, the engineered immune cell does not express the rituximab-specific mimotope of SEQ ID NO: 22.

A particular aspect concerns genetically engineered T-cells expressing a CAR22 and a CAR20 at their cell surface, wherein said CAR22 comprises the VH of SEQ ID NO: 11 and the VL of SEQ ID NO: 12, and said CAR20 comprises the VH of SEQ ID NO: 15 and the VL of SEQ ID NO: 16.

Still more particularly, are provided genetically engineered T-cells expressing a CAR22 and a CAR20 at their cell surface, wherein said CAR22 comprises the amino acid sequence of SEQ ID NO: 14, and wherein said CAR20 comprises the amino acid sequence of SEQ ID NO: 18.

A particular aspect concerns genetically engineered T-cells expressing a CAR22 and a CAR20 at their cell surface, wherein said CAR22 comprises the VH of SEQ ID NO: 11 and the VL of SEQ ID NO: 12, wherein said CAR20 comprises the VH of SEQ ID NO: 15 and the VL of SEQ ID NO: 16, and wherein said engineered T-cell has at least one allele encoding TCR alpha, TCR beta, and/or CD3 that has been inactivated by mutation.

A particular aspect concerns genetically engineered T-cells expressing a CAR22 and a CAR20 at their cell surface, wherein said CAR22 comprises the VH of SEQ ID NO: 11 and the VL of SEQ ID NO: 12, wherein said CAR20 comprises the VH of SEQ ID NO: 15 and the VL of SEQ ID NO: 16, and wherein said engineered T-cell has at least one allele encoding TCR alpha, TCR beta, and/or CD3 that has been inactivated by mutation and has at least one allele selected from CD52 and β2m that has been inactivated, in particular wherein said engineered T-cell has a TCR alpha and/or TCR beta and CD52 that have been inactivated by mutation.

Still more particularly, are provided genetically engineered T-cells expressing a CAR22 and a CAR20 at their cell surface, wherein said CAR22 comprises the amino acid sequence of SEQ ID NO: 14, wherein said CAR20 comprises the amino acid sequence of SEQ ID NO: 18, and wherein said engineered T-cell has at least one allele encoding TCR alpha, TCR beta, and/or CD3 that has been inactivated by mutation.

Still more particularly, are provided genetically engineered T-cells expressing a CAR22 and a CAR20 at their cell surface, wherein said CAR22 comprises the amino acid sequence of SEQ ID NO: 14, wherein said CAR20 comprises the amino acid sequence of SEQ ID NO: 18, and wherein said engineered T-cell has at least one allele encoding TCR alpha, TCR beta, and/or CD3 that has been inactivated by mutation and has at least one allele selected from CD52 and β2m that has been inactivated, in particular wherein said engineered T-cell has a TCR alpha and/or TCR beta and CD52 that have been inactivated by mutation.

Also provided herewith is a genetically engineered immune cell expressing a CAR22 and a CAR20 at its cell surface, as described above, wherein said CARs are encoded by an exogenous nucleic acid incorporated in said immune cell's genome and wherein said exogenous nucleic acid comprises, from 5′ to 3′:
(i) a promoter (such as the EF1alpha promoter)
(ii) a nucleic acid encoding said CAR20,
(iii) a nucleic acid encoding a self-cleaving peptide (such as P2A of SEQ ID NO: 19);
(iv) a nucleic acid encoding said CAR22,
whereby the same promoter controls the expression of said CAR20 and CAR22.

More particularly, is also provided a genetically engineered immune cell expressing a CAR22 and a CAR20 at its cell surface, as described above, wherein said exogenous nucleic acid comprises, from 5′ to 3′:
(i) a promoter (such as the EF1alpha promoter) that controls the expression of said CAR20;
(ii) a nucleic acid encoding said CAR20 comprising the signal peptide of SEQ ID NO: 1, the scFv of SEQ ID NO: 17, the CD8alpha hinge of SEQ ID NO: 4, the CD8alpha transmembrane domain of SEQ ID NO: 6, the 4-1BB co-stimulation domain of SEQ ID NO: 8, and the CD3 zeta signalisation domain of SEQ ID NO: 9);
(iii) a nucleic acid encoding a self-cleaving peptide (such as P2A of SEQ ID NO: 19);
(iv) a nucleic acid encoding said CAR22 comprising the signal peptide of SEQ ID NO: 1, the scFv of SEQ ID NO: 13, the CD8alpha hinge of SEQ ID NO: 4, the CD8alpha transmembrane domain of SEQ ID NO: 6, the 4-1BB co-stimulation domain of SEQ ID NO: 8, and the CD3 zeta signalisation domain of SEQ ID NO: 9;
whereby the same promoter controls the expression of said CAR20 and CAR22.

Alternatively, is also provided herewith a genetically engineered immune cell expressing a CAR22 and a CAR20 at its cell surface, as described above, wherein said CARs are encoded by an exogenous nucleic acid incorporated in said immune cell's genome and wherein said exogenous nucleic acid comprises, from 5′ to 3′:
(i) a promoter (such as the EF1alpha promoter)
(ii) a nucleic acid encoding said CAR22,
(iii) a nucleic acid encoding a self-cleaving peptide (such as P2A of SEQ ID NO: 19);
(iv) a nucleic acid encoding said CAR20,
whereby the same promoter controls the expression of said CAR20 and CAR22.

More particularly, is also provided a genetically engineered immune cell expressing a CAR22 and a CAR20 at its cell surface, as described above, wherein said exogenous nucleic acid comprises, from 5′ to 3′:
(i) a promoter (such as the EF1alpha promoter) that controls the expression of said CAR22;
(ii) the nucleic acid sequence encoding said CAR22 comprising the signal peptide of SEQ ID NO: 1, the scFv of SEQ ID NO: 13, the CD8alpha hinge of SEQ ID NO: 4, the CD8alpha transmembrane domain of SEQ ID NO: 6, the 4-1 BB co-stimulation domain of SEQ ID NO: 8, and the CD3 zeta signalisation domain of SEQ ID NO: 9);
(iii) a nucleic acid encoding a self-cleaving peptide (such as P2A of SEQ ID NO: 19); (iv) the nucleic acid encoding said CAR20 comprising the signal peptide of SEQ ID NO: 1, the scFv of SEQ ID NO: 17, the CD8alpha hinge of SEQ ID NO: 4, the CD8alpha transmembrane domain of SEQ ID NO: 6, the 4-1EE co-stimulation domain of SEQ ID NO: 8, and the CD3 zeta signalisation domain of SEQ ID NO: 9.

### Methods of Preparation of the Genetically Engineered Immune Cells

The immune cells to be genetically engineered to express CAR20 and CAR22 as described herewith can be prepared by introducing one or more exogenous polynucleotides encoding said CARs. Said polynucleotide can be introduced into the cell using viral vectors via transduction. Said polynucleotide can also be introduced into the cell in the form of a plasmid or a PCR product.

Stable expression of CARs, in particular the CAR20 and CAR22 described herewith, in the above-described immune cells can be achieved using, for example, viral vectors (e.g., lentiviral vectors, retroviral vectors, Adeno-Associated Virus (AAV) vectors) or transposon/transposase systems or plasmids or PCR products integration. Other approaches include direct mRNA electroporation.

To deliver both CARs simultaneously into a cell, the polynucleotide(s) encoding anti-CD22 CAR (CAR22) and anti-CD20 CAR (CAR20) described herewith can have different structures such as:
(a) a polycistronic arrangement where both of the 2 transcription units are controlled by a unique promoter, have the same direction of transcription and are separated by a “self-cleaving” peptide such as a 2A peptide (e.g. P2A, T2A, E2A, F2A),
(b) a bi-directional arrangement where each of the 2 transcription units is controlled by an independent promoter in a head to head configuration and are transcribed in opposite directions, and
(c) a monocistronic arrangement with both scFV transcripts separated by a genomic spacer.

As used herein, a “polycistronic” mRNA refers to a single messenger RNA that comprises two or more coding sequences (i.e., cistrons) and encodes more than one protein. A polycistronic mRNA can comprise any element known in the art to allow for the translation of two or more genes from the same mRNA molecule including, but not limited to, a self-cleaving peptide like a P2A element, a T2A element, an E2A element, and an F2A element, or an IRES element.

A self-cleaving peptide to be comprised in the polynucleotides described herewith can be selected from a 2A peptide, a 2A like peptide, a P2A peptide, a E2A peptide, a F2A peptide, T2A peptide, in particular a 2A peptide, more particularly a P2A peptide of SEQ ID NO: 19, a T2A peptide of SEQ ID NO: 38, a E2A peptide of SEQ ID NO: 39, or a F2A peptide of SEQ ID NO: 40, still more particularly a P2A peptide of SEQ ID NO: 19.

An IRES states for “Internal ribosome entry sites”, any IRES that allows the transcription and then translation of a coding sequence inserted in a gene could be used here. For example, an IRES comprised in the polynucleotides described herewith can have the SEQ ID NO: 37.

As described herewith the CAR20 and CAR22 can be encoded by two nucleic acids, wherein:
1) one nucleic acid encodes a CAR20 comprising:
   i) at least one extracellular domain comprising:
      an antigen binding domain specific for CD20 comprising a Variable Heavy chain (VH) comprising an amino acid sequence having at least 80%, at least 90%, at least 95%, at least 97%, at least 99%, or 100% identity with SEQ ID NO: 15 and comprising the H-CDRs of amino acid sequences SEQ ID NO: 47, SEQ ID NO: 48, and SEQ ID NO: 49, and a Variable Light chain (VL) comprising an amino acid sequence having at least 80%, at least 90%, at least 95%, at least 97%, at least 99%, or 100% identity with SEQ ID NO: 16 and comprising the L-CDRs of amino acid sequences SEQ ID NO: 50, SEQ ID NO: 51, and SEQ ID NO: 52, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain; and
2) another nucleic acid encodes a CAR22 comprising:
   i) at least one extracellular domain comprising:
      an antigen binding domain specific for CD22 comprising a Variable Heavy chain (VH) comprising an amino acid sequence having at least 80%, at least 90%, at least 95%, at least 97%, at least 99%, or 100% identity with SEQ ID NO: 11 and comprising the H-CDRs of amino acid sequences SEQ ID NO: 41, SEQ ID NO: 42, and SEQ ID NO: 43, and a Variable Light chain (VL) comprising an amino acid sequence having at least 80%, at least 90%, at least 95%, at least 97%, at least 99%, or 100% identity with SEQ ID NO: 12 and comprising the L-CDRs of amino acid sequences SEQ ID NO: 44, SEQ ID NO: 45, and SEQ ID NO: 46, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain.

In one instance, CAR22 and CAR20 are encoded by two independent nucleic acids a) and b) wherein:
(1) nucleic acid a) encodes a CAR22 comprising:
   i) at least one extracellular domain comprising:
      an antigen binding domain specific for CD22 comprising the Variable Heavy chain (VH) of SEQ ID NO: 11 and the Variable Light chain (VL) of SEQ ID NO: 12, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain; and
(2) nucleic acid b) encodes a CAR20 comprising:
   i) at least one extracellular domain comprising:
      an antigen binding domain specific for CD20 comprising the Variable Heavy chain (VH) of SEQ ID NO: 15 and the Variable Light chain (VL) of SEQ ID NO: 16, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain.

In a particular instance, is provided an isolated polynucleotide comprising:
a) a nucleic acid encoding a CAR22 comprising:
   i) at least one extracellular domain comprising:
      an antigen binding domain specific for CD22 comprising the Variable Heavy chain (VH) of SEQ ID NO: 11 and the Variable Light chain (VL) of SEQ ID NO: 12, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain; and
b) a nucleic acid encoding a CAR20 comprising:
   i) at least one extracellular domain comprising:
      an antigen binding domain specific for CD20 comprising the Variable Heavy chain (VH) of SEQ ID NO: 15 and the Variable Light chain (VL) of SEQ ID NO: 16, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain.

In a further instance, the nucleic acids of a) and b) are on a single nucleic acid molecule and the isolated polynucleotide comprises a nucleic acid sequence encoding a self-cleaving peptide (such as P2A, T2A, E2A, F2A) located between the nucleic acids of a) and b).

Thus, in a general aspect, disclosed herewith is a polynucleotide comprising, from 5′ to 3′:
(i) a promoter (such as the EF1alpha promoter)
(ii) a nucleic acid encoding a CAR20 as described herewith,
(iii) a nucleic acid encoding a self-cleaving peptide (such as P2A of SEQ ID NO: 19);
(iv) a nucleic acid encoding a CAR22 as described herewith,
whereby the same promoter controls the expression of said CAR20 and CAR22.

Also disclosed herewith is a polynucleotide comprising, from 5′ to 3′:
(i) a promoter (such as the EF1alpha promoter)
(ii) a nucleic acid encoding a CAR22 as described herewith,
(iii) a nucleic acid encoding a self-cleaving peptide (such as P2A of SEQ ID NO: 19);
(iv) a nucleic acid encoding a CAR20 as described herewith,
whereby the same promoter controls the expression of said CAR20 and CAR22.

In a particular instance, the isolated polynucleotide described herewith does not comprise a nucleic acid encoding a further CAR than said CAR22 and CAR20.

In a particular instance, the isolated polynucleotide described herewith does not comprise a nucleic acid encoding the rituximab-specific mimotope of SEQ ID NO: 22.

In one instance, the isolated polynucleotide encoding a CAR20 and CAR22 as described herewith comprises:
a) a promoter (such as the EF1alpha promoter) that controls the expression of said CAR20, followed by a nucleic acid encoding said CAR20, wherein said CAR20 comprises:
   i) at least one extracellular domain comprising:
      an antigen binding domain specific for CD20 comprising a Variable Heavy chain (VH) comprising an amino acid sequence having at least 80%, at least 90%, at least 95%, at least 97%, at least 99%, or 100% identity with SEQ ID NO: 15 and comprising the H-CDRs of amino acid sequences SEQ ID NO: 47, SEQ ID NO: 48, and SEQ ID NO: 49, and a Variable Light chain (VL) comprising an amino acid sequence having at least 80%, at least 90%, at least 95%, at least 97%, at least 99%, or 100% identity with SEQ ID NO: 16 and comprising the L-CDRs of amino acid sequences SEQ ID NO: 50, SEQ ID NO: 51, and SEQ ID NO: 52, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain; and
b) a nucleic acid encoding a CAR22, wherein said CAR22 comprises:
   i) at least one extracellular domain comprising:
      an antigen binding domain specific for CD22 comprising a Variable Heavy chain (VH) comprising an amino acid sequence having at least 80%, at least 90%, at least 95%, at least 97%, at least 99%, or 100% identity with SEQ ID NO: 11 and comprising the H-CDRs of amino acid sequences SEQ ID NO: 41, SEQ ID NO: 42, and SEQ ID NO: 43, and a Variable Light chain (VL) comprising an amino acid sequence having at least 80%, at least 90%, at least 95%, at least 97%, at least 99%, or 100% identity with SEQ ID NO: 12 and comprising the L-CDRs of amino acid sequences SEQ ID NO: 44, SEQ ID NO: 45, and SEQ ID NO: 46, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain;
wherein said nucleic acids of a) and b) are on a single nucleic acid molecule and wherein a nucleic acid sequence encoding a self-cleaving peptide (such as P2A, T2A, E2A, or F2A) is located between said nucleic acids of a) and b).

In an alternative instance, the isolated polynucleotide encoding a CAR20 and CAR22 as described herewith comprises:
a) a promoter (such as the EF1alpha promoter) that controls the expression of said CAR22, followed by a nucleic acid encoding said CAR22, wherein said CAR22 is composed of:
   i) at least one extracellular domain comprising:
      an antigen binding domain specific for CD22 comprising a Variable Heavy chain (VH) comprising an amino acid sequence having at least 80%, at least 90%, at least 95%, at least 97%, at least 99%, or 100% identity with SEQ ID NO: 11 and comprising the H-CDRs of amino acid sequences SEQ ID NO: 41, SEQ ID NO: 42, and SEQ ID NO: 43, and a Variable Light chain (VL) comprising an amino acid sequence having at least 80%, at least 90%, at least 95%, at least 97%, at least 99%, or 100% identity with SEQ ID NO: 12 and comprising the L-CDRs of amino acid sequences SEQ ID NO: 44, SEQ ID NO: 45, and SEQ ID NO: 46, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain; and
b) a nucleic acid encoding a CAR20, wherein said CAR20 comprises:
   i) at least one extracellular domain comprising:
      an antigen binding domain specific for CD20 comprising a Variable Heavy chain (VH) comprising an amino acid sequence having at least 80%, at least 90%, at least 95%, at least 97%, at least 99%, or 100% identity with SEQ ID NO: 15 and comprising the H-CDRs of amino acid sequences SEQ ID NO: 47, SEQ ID NO: 48, and SEQ ID NO: 49, and a Variable Light chain (VL) comprising an amino acid sequence having at least 80%, at least 90%, at least 95%, at least 97%, at least 99%, or 100% identity with SEQ ID NO: 16 and comprising the L-CDRs of amino acid sequences SEQ ID NO: 50, SEQ ID NO: 51, and SEQ ID NO: 52, optionally a leader sequence,
      a hinge domain from CD8alpha,
   ii) a transmembrane domain from CD8alpha, and
   iii) an intracellular domain comprising a 4-1BB stimulatory domain and a CD3zeta signalling domain;
wherein said nucleic acids of a) and b) are on a single nucleic acid molecule and wherein a nucleic acid sequence encoding a self-cleaving peptide (such as P2A, T2A, E2A, or F2A) is located between said nucleic acids of a) and b).

In a still further instance, the isolated polynucleotide encoding a CAR20 and CAR22 as described herewith comprises:
a) a nucleic acid encoding a CAR22, comprising a promoter (such as the EF1alpha promoter) that controls the expression of said CAR22 composed of the signal peptide of SEQ ID NO: 1, the scFv of SEQ ID NO: 13, the CD8alpha hinge of SEQ ID NO: 4, the CD8alpha transmembrane domain of SEQ ID NO: 6, the 4-11BE co-stimulation domain of SEQ ID NO: 8, and the CD3 zeta signalisation domain of SEQ ID NO: 9;
b) a nucleic acid encoding a CAR20 composed of the signal peptide of SEQ ID NO: 1, the scFv of SEQ ID NO: 17, the CD8alpha hinge of SEQ ID NO: 4, the CD8alpha transmembrane domain of SEQ ID NO: 6, the 4-11BE co-stimulation domain of SEQ ID NO: 8, and the CD3 zeta signalisation domain of SEQ ID NO: 9; and
c) a nucleic acid encoding a self-cleaving peptide (such as P2A of SEQ ID NO: 19) placed between the nucleic acid of a) and the nucleic acid of b), allowing the simultaneous expression of said CAR20 and CAR22.

In a still further instance, is provided the isolated polynucleotide encoding a CAR20 and CAR22 as described herewith comprises:
a) a nucleic acid encoding a CAR20, comprising a promoter (such as the EF1alpha promoter) that controls the expression of said CAR20 composed of the signal peptide of SEQ ID NO: 1, the scFv of SEQ ID NO: 17, the CD8alpha hinge of SEQ ID NO: 4, the CD8alpha transmembrane domain of SEQ ID NO: 6, the 4-11BE co-stimulation domain of SEQ ID NO: 8, and the CD3 zeta signalisation domain of SEQ ID NO: 9;
b) a nucleic acid encoding a CAR22 composed of the signal peptide of SEQ ID NO: 1, the scFv of SEQ ID NO: 13, the CD8alpha hinge of SEQ ID NO: 4, the CD8alpha transmembrane domain of SEQ ID NO: 6, the 4-1 BB co-stimulation domain of SEQ ID NO: 8, and the CD3 zeta signalisation domain of SEQ ID NO: 9; and
c) a nucleic acid encoding a self-cleaving peptide (such as P2A of SEQ ID NO: 19) placed between the nucleic acid of a) and the nucleic acid of b), allowing the simultaneous expression of said CAR22 and CAR20.

In a still further instance is provided an isolated polynucleotide encoding a CAR20 and CAR22 comprising the nucleic acid sequence of SEQ ID NO: 31 (CAR22xCAR20 construct).

In another particular instance, is provided an isolated polynucleotide encoding a CAR20 and CAR22 comprising the nucleic acid sequence of SEQ ID NO: 32 (CAR20xCAR22 construct).

Also disclosed herewith is a vector comprising any of the isolated polynucleotides described herewith.

Also disclosed herewith is a host cell comprising the vector described herewith.

A population of immune cells to be engineered is generally extracted from a patient's or healthy donor's blood by apheresis and further engineered to express a chimeric antigen receptor at their surface. Alternatively, a population of immune cells to be engineered could derive from cord blood cells or from stem cells, which are further engineered to express a chimeric antigen receptor at their surface.

Said CAR-expressing immune cells may derive from a patient's or a compatible donor's immune cells which have been engineered to express a specific CAR at their surface.

Said CAR-expressing immune cells can also derive from stem cells, such as PS cells, originating from such patient or compatible donor or from tumor infiltrating lymphocytes (TILL).

In other aspects, said CAR-expressing immune cells are so-called “off the shelf” immune cells compositions, whereby immune cells not specially belonging to the patient to be treated have been engineered to express a CAR and to become suitable for use in an allogeneic therapeutic treatment.

By “allogeneic” is meant that the cells originate from a donor, or are produced and/or differentiated from stem cells in view of being infused into patients having a different haplotype.

Such immune cells are generally engineered to be less alloreactive and/or become more persistent with respect to their patient host. More specifically, the method of engineering allogeneic immune cells can comprise the step of reducing or inactivating TCR expression into T-cells, or into the stem cells to be derived into T-cells. This can be obtained by different sequence specific-reagents, such as by gene silencing or gene editing techniques (nuclease, base editing, RNAi . . . ).

The applicant has formerly made available robust protocols and gene editing strategies to produce allogeneic therapeutic grade T-cells from PBMCs, especially by providing very safe and specific endonuclease reagents under the form of TALE-nucleases (TALEN®). The production of so-called “universal T-cells”, which are [TCR]^{negative} T-cells from donors was achieved and successfully injected to patients with reduced Graft versus Host Disease (GVhD) (Poirot et al. 2015, Cancer. Res. 75 (18): 3853-3864; Qasim et al., 2017, Science Translational 9(374)). Meanwhile, inactivation of TCR or β2m components in primary T-cells can be combined with the inactivation of further genes encoding checkpoint inhibitor proteins, such as described for instance in WO2014184744.

In further instances, the engineered immune cell can be further modified to confer resistance to at least one immune suppressive drug, such as by inactivating CD52 that is the target of anti-CD52 antibody (e.g.: alemtuzumab), as described for instance in WO2013176915.

In further instances, the engineered immune cell can be further modified to confer resistance to and/or a chemotherapy drug, in particular a purine analogue drug, for example by inactivating DCK as described in WO201575195.

In further instances, the engineered immune cell can be further modified to improve its persistence or its lifespan into the patient, in particular inactivating a gene encoding MHC-I component(s) such as HLA or β2m, such as described in WO2015136001 or by Liu et al. (2017, Cell Res 27:154-157).

In still further instances, the engineered immune cell is mutated to improve its CAR-dependent immune activation, in particular to reduce or suppress the expression of immune checkpoint proteins and/or their receptors thereof, such as PD1 or CTLA4 as described in WO2014184744.

### Pharmaceutical Compositions

One aspect relates to a pharmaceutical composition comprising the genetically engineered immune cells expressing a Chimeric Antigen Receptor (CAR) specific for CD22 (CAR22) and a Chimeric Antigen Receptor specific for CD20 (CAR20) at their cell surface as described herewith, and a pharmaceutically acceptable excipient.

Also disclosed is a pharmaceutical composition comprising a population of immune cells comprising genetically engineered immune cells expressing a Chimeric Antigen Receptor (CAR) specific for CD22 (CAR22) and a Chimeric Antigen Receptor specific for CD20 (CAR20) at their cell surface as described herewith, and a pharmaceutical acceptable excipient.

A particular aspect concerns a pharmaceutical composition comprising the genetically engineered T-cells as described herewith and a pharmaceutically acceptable excipient.

A further particular aspect relates to a pharmaceutical composition comprising a population of T-cells comprising the genetically engineered T-cells as described herewith and a pharmaceutically acceptable excipient.

Another particular aspect concerns a pharmaceutical composition comprising genetically engineered T-cells expressing a CAR22 and a CAR20 at their cell surface and a pharmaceutical excipient, wherein said CAR22 comprises the VH of SEQ ID NO: 11 and the VL of SEQ ID NO: 12, and said CAR20 comprises the VH of SEQ ID NO: 15 and the VL of SEQ ID NO: 16.

Still more particularly, is provided a pharmaceutical composition comprising genetically engineered T-cells expressing a CAR22 and a CAR20 at their cell surface and a pharmaceutical excipient, wherein said CAR22 comprises the amino acid sequence of SEQ ID NO: 14, and wherein said CAR20 comprises the amino acid sequence of SEQ ID NO: 18.

A further particular aspect relates to a pharmaceutical composition comprising a population of T-cells comprising genetically engineered T-cells expressing a CAR22 and a CAR20 at their cell surface and a pharmaceutical excipient, wherein said CAR22 comprises the VH of SEQ ID NO: 11 and the VL of SEQ ID NO: 12, and said CAR20 comprises the VH of SEQ ID NO: 15 and the VL of SEQ ID NO: 16.

A further particular aspect relates to a pharmaceutical composition comprising a population of T-cells comprising genetically engineered T-cells expressing a CAR22 and a CAR20 at their cell surface and a pharmaceutical excipient, wherein said CAR22 comprises the amino acid sequence of SEQ ID NO: 14, and wherein said CAR20 comprises the amino acid sequence of SEQ ID NO: 18.

Another particular aspect concerns a pharmaceutical composition comprising genetically engineered T-cells expressing a CAR22 and a CAR20 at their cell surface and a pharmaceutical excipient, wherein said CAR22 comprises the VH of SEQ ID NO: 11 and the VL of SEQ ID NO: 12, and said CAR20 comprises the VH of SEQ ID NO: 15 and the VL of SEQ ID NO: 16, and wherein said T-cells have at least one allele encoding TCR alpha, TCR beta, and/or CD3 that has been inactivated by mutation, and/or wherein said engineered T-cells have at least one allele selected from β2m and CD52 that has been inactivated.

Still more particularly, is provided a pharmaceutical composition comprising genetically engineered T-cells expressing a CAR22 and a CAR20 at their cell surface and a pharmaceutical excipient, wherein said CAR22 comprises the amino acid sequence of SEQ ID NO: 14, wherein said CAR20 comprises the amino acid sequence of SEQ ID NO: 18, and wherein said T-cells have at least one allele encoding TCR alpha, TCR beta, and/or CD3 that has been inactivated by mutation.

Still more particularly, is provided a pharmaceutical composition comprising genetically engineered T-cells expressing a CAR22 and a CAR20 at their cell surface and a pharmaceutical excipient, wherein said CAR22 comprises the amino acid sequence of SEQ ID NO: 14, wherein said CAR20 comprises the amino acid sequence of SEQ ID NO: 18, and wherein said T-cells have at least one allele encoding TCR alpha, TCR beta, and/or CD3 that has been inactivated by mutation, and/or wherein said engineered T-cells have at least one allele selected from β2m and CD52 that has been inactivated.

A further particular aspect relates to a pharmaceutical composition comprising a population of T-cells comprising genetically engineered T-cells expressing a CAR22 and a CAR20 at their cell surface and a pharmaceutical excipient, wherein said CAR22 comprises the VH of SEQ ID NO: 11 and the VL of SEQ ID NO: 12, said CAR20 comprises the VH of SEQ ID NO: 15 and the VL of SEQ ID NO: 16, and wherein said T-cells have at least one allele encoding TCR alpha, TCR beta, and/or CD3 that has been inactivated by mutation, and/or wherein said engineered T-cells have at least one allele selected from β2m and CD52 that has been inactivated.

A further particular aspect relates to a pharmaceutical composition comprising a population of T-cells comprising genetically engineered T-cells expressing a CAR22 and a CAR20 at their cell surface and a pharmaceutical excipient, wherein said CAR22 comprises the amino acid sequence of SEQ ID NO: 14, wherein said CAR20 comprises the amino acid sequence of SEQ ID NO: 18, and wherein said T-cells have at least one allele encoding TCR alpha, TCR beta, and/or CD3 that has been inactivated by mutation.

A further particular aspect relates to a pharmaceutical composition comprising a population of T-cells comprising genetically engineered T-cells expressing a CAR22 and a CAR20 at their cell surface and a pharmaceutical excipient, wherein said CAR22 comprises the amino acid sequence of SEQ ID NO: 14, wherein said CAR20 comprises the amino acid sequence of SEQ ID NO: 18, and wherein said T-cells have at least one allele encoding TCR alpha, TCR beta, and/or CD3 that has been inactivated by mutation, and/or wherein said engineered T-cells have at least one allele selected from β2m and CD52 that has been inactivated.

Also provided herewith are genetically engineered immune cells expressing CAR20 and CAR22 as described herewith for use as a medicament.

Also provided herewith is a population of immune cells comprising the genetically engineered immune cells expressing CAR20 and CAR22 as described herewith for use as a medicament.

### Methods of Treatment

Another aspect relates to methods of treatment of a cancer and/or an inflammatory disorder comprising administering, to a patient in need thereof, the genetically engineered immune cells expressing CAR20 and CAR22 as described herewith.

A similar aspect concerns the genetically engineered immune cells expressing CAR20 and CAR22 as described herewith, for use in a method of treatment of a cancer and/or inflammatory disorder.

A similar aspect concerns a population of immune cells comprising the genetically engineered immune cells expressing CAR20 and CAR22 as described herewith, for use in a method of treatment of a cancer and/or inflammatory disorder.

A similar aspect concerns the use of the genetically engineered immune cells expressing CAR20 and CAR22 as described herewith, or the population of immune cells comprising the genetically engineered immune cells expressing CAR20 and CAR22 as described herewith, for the preparation of a medicament.

A similar aspect concerns the use of the genetically engineered immune cells expressing CAR20 and CAR22 as described herewith, or the population of immune cells comprising the genetically engineered immune cells expressing CAR20 and CAR22 as described herewith, for the preparation of a medicament for treating a cancer and/or an inflammatory disorder.

The treatment may be for treating cancers including a hematological cancer such as a hematological cancer selected from (lymphoma, Hodgkin lymphoma (HL), non-Hodgkin lymphoma (NHL), leukemia, multiple myeloma (MM), B-chronic lymphocytic leukemia (B-CLL), hairy cell leukemia (HCL), acute lymphocytic leukemia (ALL) (also known as acute lymphoblastic leukemia), acute lymphocytic cancer, acute myeloid leukemia (AML), in particular a CD22- and/or CD20-associated hematological cancer, more particularly a relapse refractory CD22- and/or CD20-associated hematological cancer, even more particularly an aggressive form of said CD22- and/or CD20-associated hematological cancer.

The treatment may relate to the prevention or attenuation of an inflammatory disorder associated with CD20 and/or CD22.

In a particular aspect, the treatment is for treating patients with relapse refractory NHL.

In a further particular aspect, the treatment is for treating patients suffering from cancers where the expression of CD20 and CD22 antigens is low.

In a particular aspect, the treatment is for treating patients having been previously treated with Rituximab (anti-CD20 antibody that is a standard of care in the treatment of NHL).

“Non-Hodgkin lymphoma (NHL)” is the term for a diverse group of blood cancers that share a single characteristic: they all arise from lymphocytes. More than 60 specific NHL subtypes have been identified and assigned names, called “diagnostic designations,” by the World Health Organization (WHO).

In particular, the term “Non-Hodgkin Lymphoma (NHL)” includes the following diagnostic designations for Non-Hodgkin Lymphoma (NHL):
1. Mature B-cell lymphomas (about 85%-90% of NHL cases):
   Aggressive: Diffuse large B-cell lymphoma (DLBCL) (31%), Mantle cell lymphoma (MCL) (can present as aggressive or indolent) (6%), Lymphoblastic lymphoma (2%), Burkitt lymphoma (BL) (2%), Primary mediastinal (thymic) large B-cell lymphoma (PMBCL) (2%), Transformed follicular and transformed mucosa-associated lymphoid tissue (MALT) lymphomas, High-grade B-cell lymphoma with double or triple hits (HBL), Primary cutaneous DLBCL (leg type), Primary DLBCL of the central nervous system, Primary central nervous system (CNS) lymphoma, Acquired immunodeficiency syndrome (AIDS)-associated lymphoma
   Indolent: Follicular lymphoma (FL) (22%), Marginal zone lymphoma (MZL) (8%), Chronic lymphocytic leukemia/small-cell lymphocytic lymphoma (CLL/SLL) (6%), Gastric mucosa-associated lymphoid tissue (MALT) lymphoma (5%), Lymphoplasmacytic lymphoma (1%), Waldenström macroglobulinemia (WM), Nodal marginal zone lymphoma (NMZL) (1%), Splenic marginal zone lymphoma (SMZL),
2. Mature T-cell and natural killer (NK)-cell lymphomas (about 10%-15% of NHL cases)
   Aggressive: Peripheral T-cell lymphoma (PTCL), not otherwise specified (6%), Systemic anaplastic large-cell lymphoma (ALCL) (2%), Lymphoblastic lymphoma (2%), Hepatosplenic gamma/delta T-cell lymphoma, Subcutaneous panniculitis-like T-cell lymphoma (SPTCL), Enteropathy-type intestinal T-cell lymphoma, Primary cutaneous anaplastic large-cell lymphoma
   Indolent: Cutaneous T-cell lymphoma (CTCL) (4%), Mycosis fungoides (MF), Sezary syndrome (SS), Angioimmunoblastic T-cell lymphoma (AITL), Adult T-cell leukemia/lymphoma, Extranodal NK/T-cell lymphoma (ENK/TCL), nasal type.

The above written description of the invention provides a manner and process of making and using it such that any person skilled in this art is enabled to make and use the same, this enablement being provided in particular for the subject matter of the appended claims, which make up a part of the original description.

Where a numerical limit or range is stated herein, the endpoints are included. Also, all values and subranges within a numerical limit or range are specifically included as if explicitly written out.

Having generally described this invention, a further understanding can be obtained by reference to certain specific examples, which are provided herein for purposes of illustration only, and are not intended to limit the scope of the claimed invention.

### Particular Embodiments

1. A genetically engineered T-cell expressing a Chimeric Antigen Receptor (CAR) specific for CD22 (CAR22) and a Chimeric Antigen Receptor specific for CD20 (CAR20) at its cell surface,
   a) wherein said CAR22 comprises:
      i) at least one extracellular domain comprising:
         an antigen binding domain specific for CD22 comprising the Variable Heavy chain (VH) of SEQ ID NO: 11 and the Variable Light chain (VL) of SEQ ID NO: 12, optionally a leader sequence,
         a hinge domain from CD8alpha,
      ii) a transmembrane domain from CD8alpha, and
      iii an intracellular domain comprising a 4-1BB stimulatory domain and a CD3zeta signalling domain; and
   b) wherein said CAR20 comprises:
      i) at least one extracellular domain comprising:
         an antigen binding domain specific for CD20 comprising the Variable Heavy chain (VH) of SEQ ID NO: 15 and the Variable Light chain (VL) of SEQ ID NO: 16, optionally a leader sequence,
         a hinge domain from CD8alpha,
      ii) a transmembrane domain from CD8alpha, and
      iii) an intracellular domain comprising a 4-1BB stimulatory domain and a CD3zeta signalling domain; and
   c) wherein said engineered T-cell is TCR negative.
2. The genetically engineered T-cell according to embodiment 1, wherein said CAR22 comprises the amino acid sequence of SEQ ID NO: 14, and wherein said CAR20 comprises the amino acid sequence of SEQ ID NO: 18.
3. The genetically engineered T-cell according to any one of embodiments 1 to 2, wherein none of the extracellular domain of said CAR20 and CAR22 comprises a rituximab-specific mimotope of SEQ ID NO: 22.
4. The genetically engineered T-cell of any one of embodiments 1 to 3, wherein said engineered T-cell expresses a short hairpin RNA (shRNA) or small interfering (siRNA) directed against a polynucleotide sequence encoding a component of TCR.
5. The genetically engineered T-cell of any one of embodiments 1 to 3, wherein said engineered T-cell is mutated in its TCR alpha and/or TCR beta alleles.
6. The genetically engineered T-cell according to any one of embodiments 1 to 3, and 5, wherein said engineered T-cell has at least one allele encoding TCR alpha, TCR beta, and/or CD3 that has been inactivated by mutation.
7. The genetically engineered T-cell according to any one of embodiments 1 to 6, wherein said engineered T-cell has a CD52 allele that has been inactivated.
8. The genetically engineered T-cell according to any one of embodiments 1 to 7, wherein said engineered T-cell has a β2m allele that has been inactivated.
9. The genetically engineered T-cell according to any one of embodiments 1 to 8, wherein said engineered T-cell has a PD1 allele that has been inactivated.
10. The genetically engineered T-cell according to any one of embodiments 1 to 9, wherein said engineered T-cell has a CTLA4 allele that has been inactivated.
11. The genetically engineered T-cell according to any one of embodiments 1 to 10, wherein said engineered T-cell has a dCK allele that has been inactivated.
12. The genetically engineered T-cell according to any one of embodiments 1 to 11, wherein said engineered T-cell has a GR allele that has been inactivated.
13. The genetically engineered T-cell according to any one of embodiments 1 to 12, wherein said engineered immune cell is a cytotoxic T cell.
14. The genetically engineered T-cell according to any one of embodiments 1 to 13, wherein said engineered T-cell is comprised in a population of T-cells.
15. The genetically engineered T-cell according to any one of embodiments 1 to 14, wherein said engineered T-cell is a primary cell.
16. The genetically engineered T-cell according to any one of embodiments 1 to 15, wherein said T-cell is a mammalian cell, preferably a human cell.
17. The genetically engineered T-cell according to to any one of embodiments 1 to 16, wherein said engineered T-cell expresses no further CAR than said CAR22 and CAR20.
18. A population of T-cells comprising the engineered T-cells according to any one of embodiments 1 to 17.
19. A pharmaceutical composition comprising the engineered T-cells according to any one of embodiments 1 to 17, and a pharmaceutically acceptable excipient.
20. A pharmaceutical composition comprising a population of T-cells according to embodiment 18, and a pharmaceutically acceptable excipient.
21. An isolated polynucleotide comprising:
   a) a nucleic acid encoding a CAR22 comprising:
      i) at least one extracellular domain comprising:
         an antigen binding domain specific for CD22 comprising the Variable Heavy chain (VH) of SEQ ID NO: 11 and the Variable Light chain (VL) of SEQ ID NO: 12, optionally a leader sequence,
         a hinge domain from CD8alpha,
      ii) a transmembrane domain from CD8alpha, and
      iii) an intracellular domain comprising a 4-1BB stimulatory domain and a CD3zeta signalling domain; and
   b) a nucleic acid encoding a CAR20 comprising:
      i) at least one extracellular domain comprising:
         an antigen binding domain specific for CD20 comprising the Variable Heavy chain (VH) of SEQ ID NO: 15 and the Variable Light chain (VL) of SEQ ID NO: 16, optionally a leader sequence,
         a hinge domain from CD8alpha,
      ii) a transmembrane domain from CD8alpha, and
      iii) an intracellular domain comprising a 4-1BB stimulatory domain and a CD3zeta signalling domain.
22. The polynucleotide of embodiment 21, wherein the nucleic acids of a) and b) are on a single nucleic acid molecule and wherein a nucleic acid sequence encoding a self-cleaving peptide (such as P2A, T2A, E2A, or F2A) is located between the nucleic acids of a) and b).
23. The polynucleotide of embodiment 21 or 22, wherein said isolated polynucleotide does not comprise a nucleic acid encoding a further CAR than said CAR22 and CAR20.
24. The polynucleotide of embodiment 21 to 23, wherein said isolated polynucleotide does not comprise a nucleic acid encoding rituximab-specific mimotope of SEQ ID NO: 22.
25. The polynucleotide of any one of embodiments 21 to 24, wherein the nucleic acid of a) comprises a promoter (such as the EF1alpha promoter) that controls the expression of said CAR22 composed of the signal peptide of SEQ ID NO: 1, the scFv of SEQ ID NO: 13, the CD8alpha hinge of SEQ ID NO: 4, the CD8alpha transmembrane domain of SEQ ID NO: 6, the 4-11BE co-stimulation domain of SEQ ID NO: 8, and the CD3 zeta signalisation domain of SEQ ID NO: 9;
   wherein the nucleic acid of b) encodes for said CAR20 composed of the signal peptide of SEQ ID NO: 1, the scFv of SEQ ID NO: 17, the CD8alpha hinge of SEQ ID NO: 4, the CD8alpha transmembrane domain of SEQ ID NO: 6, the 4-1BB co-stimulation domain of SEQ ID NO: 8, and the CD3 zeta signalisation domain of SEQ ID NO: 9; and wherein a nucleic acid encoding for a self-cleaving peptide (such as P2A of SEQ ID NO: 19) is present between the nucleic acid of a) and the nucleic acid of b), allowing the simultaneous expression of said CAR20 and CAR22.
26. The polynucleotide of any one of embodiments 21 to 24, wherein the nucleic acid of b) comprises a promoter (such as the EF1alpha promoter) that controls the expression of said CAR20 composed of the signal peptide of SEQ ID NO: 1, the scFv of SEQ ID NO: 17, the CD8alpha hinge of SEQ ID NO: 4, the CD8alpha transmembrane domain of SEQ ID NO: 6, the 4-11BE co-stimulation domain of SEQ ID NO: 8, and the CD3 zeta signalisation domain of SEQ ID NO: 9;
   wherein the nucleic acid of a) encodes for said CAR22 composed of the signal peptide of SEQ ID NO: 1, the scFv of SEQ ID NO: 13, the CD8alpha hinge of SEQ ID NO: 4, the CD8alpha transmembrane domain of SEQ ID NO: 6, the 4-1EE co-stimulation domain of SEQ ID NO: 8, and the CD3 zeta signalisation domain of SEQ ID NO: 9; and wherein a nucleic acid encoding for a self-cleaving peptide (such as P2A of SEQ ID NO: 19) is present between the nucleic acid of a) and the nucleic acid of b), allowing the simultaneous expression of said CAR22 and CAR20.
27. The isolated polynucleotide of embodiment 25 comprising the nucleic acid sequence of SEQ ID NO: 31 (CAR22xCAR20 construct).
28. The isolated polynucleotide of embodiment 26 comprising the nucleic acid sequence of SEQ ID NO: 32 (CAR20xCAR22 construct).
29. A vector comprising the isolated polynucleotide of any one of embodiments 21 to 28.
30. A host cell comprising the vector of embodiment 29.
31. A method of preparing the engineered T-cells according to any one of embodiments 1 to 17, comprising introducing into an immune cell the polynucleotide according to any one of embodiments 21 to 28, or a vector according to embodiment 29.
32. The engineered T-cells according to any one of embodiments 1 to 17, for use as a medicament.
33. The engineered T-cells according to any one of embodiments 1 to 17, for use in the treatment of a cancer associated with CD20 and/or CD22 expression.
34. The engineered T-cells according to any one of embodiments 1 to 17, for use in the treatment of a hematological cancer, in particular a CD22- and/or CD20-associated hematological cancer, more particularly a relapse refractory CD22- and/or CD20-associated hematological cancer, even more particularly an aggressive form of said CD22- and/or CD20-associated hematological cancer.
35. The engineered T-cells for use according to embodiment 33 or 34, wherein said cancer is selected from the group consisting of lymphoma, Hodgkin lymphoma (HL), non-Hodgkin lymphoma (NHL), leukemia, multiple myeloma (MM), B-chronic lymphocytic leukemia (B-CLL), hairy cell leukemia (HCL), acute lymphocytic leukemia (ALL), acute lymphocytic cancer, acute myeloid leukemia (AML).
36. The engineered T-cells for use according to embodiment 35, wherein said cancer is non-Hodgkin lymphoma or acute lymphocytic leukemia.
37. The engineered T-cells for use according to any one of embodiments 33 to 36, wherein said cancer is associated with a low expression of CD20 and/or CD22.
38. The engineered T-cells for use according to any one of embodiments 33 to 37, wherein said cancer is a relapsing non-Hodgkin lymphoma.
39. A method of treating a patient suffering from a cancer associated with CD20 and/or CD22 expression, comprising administering to said patient an effective amount of an engineered T-cells according to any one of embodiments 1 to 17, or a population of T-cells according to embodiment 18.
40. The method of treatment according to embodiment 39, wherein said cancer is selected from the group consisting of lymphoma, Hodgkin lymphoma (HL), non-Hodgkin lymphoma (NHL), leukemia, multiple myeloma (MM), B-chronic lymphocytic leukemia (B-CLL), hairy cell leukemia (HCL), acute lymphocytic leukemia (ALL), acute lymphocytic cancer, acute myeloid leukemia (AML).
41. The method of treatment according to embodiment 39 or 40, wherein said cancer is non-Hodgkin lymphoma or acute lymphocytic leukemia.
42. The method of treatment according to any one of embodiments 39 to 41, wherein said cancer is associated with a low expression of CD20 and/or CD22.
43. The method of treatment according to any one of embodiments 39 to 42, wherein said cancer is a relapsing non-Hodgkin lymphoma.

### EXAMPLES

The examples provided herewith describe how to generate dual CAR-T cells targeting CD20 and CD22 antigens (“CD20xCD22” or “CD20xCD22” CAR-T cells) and demonstrate their ability to lyse CD22 and/or CD20 low- or not-expressing tumor cells.

### Example 1. Generation of the Dual CD20 and CD22 CAR Constructs and Their Control

Two orientations of the dual CAR were designed and constructed into recombinant lentiviral vector in order to be compared for expression, activity and efficiency. The first construct (CD22xCD20) contains the EF1alpha promoter that drives the expression of a first CD22 CAR composed of a signal peptide (SEQ ID NO: 1), the scFv of SEQ ID NO: 13, CD8alpha hinge (SEQ ID NO: 4) and TM domain (SEQ ID NO: 6), 4-1 BB co-stimulation domain (SEQ ID NO: 8) and the CD3 zeta signalisation domain (SEQ ID NO: 9). This first CAR is followed by a self-cleaving peptide P2A (SEQ ID NO: 19) allowing the expression of the second CD20 CAR composed of signal peptide (SEQ ID NO:1), the anti-CD20 scFv of SEQ ID NO: 17, CD8alpha hinge (SEQ ID NO: 4) and TM domain (SEQ ID NO: 6), 4-1EE co-stimulation domain (SEQ ID NO: 8), and the CD3 zeta signalisation domain (SEQ ID NO: 9). In order to avoid any recombination event within the rLV construct, the nucleotide acid sequences used to code for the same amino acid sequences present twice in the construct were optimized using codon usage and code degeneracy so that the nucleotide sequences diverge. The second dual construct (CD20xCD22) contains exactly the same sequences (nucleotide acid and amino acid) but the CD20 CAR is located right after the EF1alpha promoter.

For comparison CD22 or CD20 CARs were constructed into recombinant lentiviral vector. Those constructs contain EF1alpha promoter driving the expression of the single CAR targeting CD22 (SEQ ID NO: 14) or CD20 (SEQ ID NO: 18).

### Example 2: Generation of CD20xCD22 or CD22xCD20 Dual CAR-T Cells

Cryopreserved PBMC from at least 3 different donors were used. PBMCs were thawed at 37° C., washed and re-suspended in OpTmizer medium supplemented with AB human serum (5%) recombinant human interleukin-2 (rhIL-2, 350 IU/ml) for an overnight incubation at 37° C. in 5% CO₂ incubator. Then, the cells were activated with anti-CD3/CD28 coated beads in OpTmizer medium supplemented with AB human serum (5%) (and additional 5% CTS™ Immune Cell SR during the expansion phase) and recombinant human interleukin-2 (rhlL-2, 350 IU/ml) in a CO₂ incubator for 3 days. The amplified T-cells were then transduced with lentiviral particles expressing CD20xCD22 or CD22xCD20 CARs (SEQ ID NO: 31 and SEQ ID NO: 32, respectively) at MOI 15 (MOI stands for Multiplicity of Infection) in the presence of Lentiboost (Mayflower bioscience SB-P-LV-101-12). The amplified T-cells were also transduced with lentiviral particles expressing either CD20 CAR or CD22 CAR alone (SEQ ID NO: 14 and SEQ ID NO: 18) at MOI 5. Two days post transduction cells were electroporated with 4 mRNAs, two encoding of TRAC_T01 TALEN arms (SEQ ID NO: 23 and SEQ ID NO: 24) and two encoding CD52_T01 TALEN arms (SEQ ID NO: 25 and SEQ ID NO: 26) using AgilePulse Max system. Cells were resuspended in culture medium incubated 16 to 18 h at 30° C. and expanded at 37° C. after addition of fresh culture medium and adjusting cell concentration from time to time. On the final day of culture (18 days post thawing), T cells were used in different assays or frozen in freezing medium (FBS 90%, DMSO 10%). Cells were kept frozen at −150° C. until use.

### Example 3. CD20 and CD22 Dual CAR-T Cells Detection

In order to detect the CD20 CAR, a recombinant protein CD20 fused to a His tag (Acro #CD0-H52H3, SEQ ID NO: 20) was used coupled to APC labelled anti-His antibody (BioLegend #362605). For the CD22 CAR detection, a CD22-Fc protein (SEQ ID NO: 21) was used coupled with an anti-Fcgamma sub class 1 tag Cy3 (Jackson ImmunoResearch #115-165-205).

The different CAR T cells (untransduced, CD20xCD22 CAR, CD22xCD20 CAR or CD22 CAR) produced in example 1 were incubated with 100 ng of CD22-Fc protein and 200 ng of CD20-His protein, washed and further incubated with anti-Fc gamma (50 ng) or anti-His (50 ng) then fixed in PFA 2%. Cells were then analysed by flow cytometry.

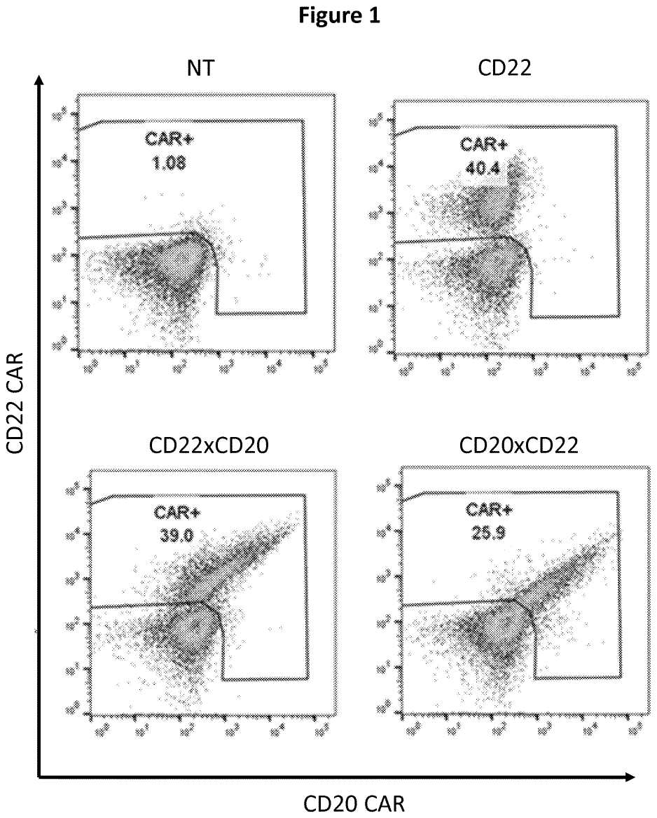

The results in FIG. 1 demonstrate that transduction with CD22 CAR alone led to 40% of CD22 CAR positive T cells whereas transduction with CD22xCD20 or CD20xCD22 CAR led to 39% and 25% of double CAR positive T cells, respectively. These results were reproduced using three different donors. Interestingly CD22xCD20 led to a higher MFI of the CD22 CAR staining whereas CD20xCD22 led to a higher MFI of the CD20 CAR staining.

### Example 4. Cell Lines Used for Testing CD20 and CD22 Dual CAR-T Cells

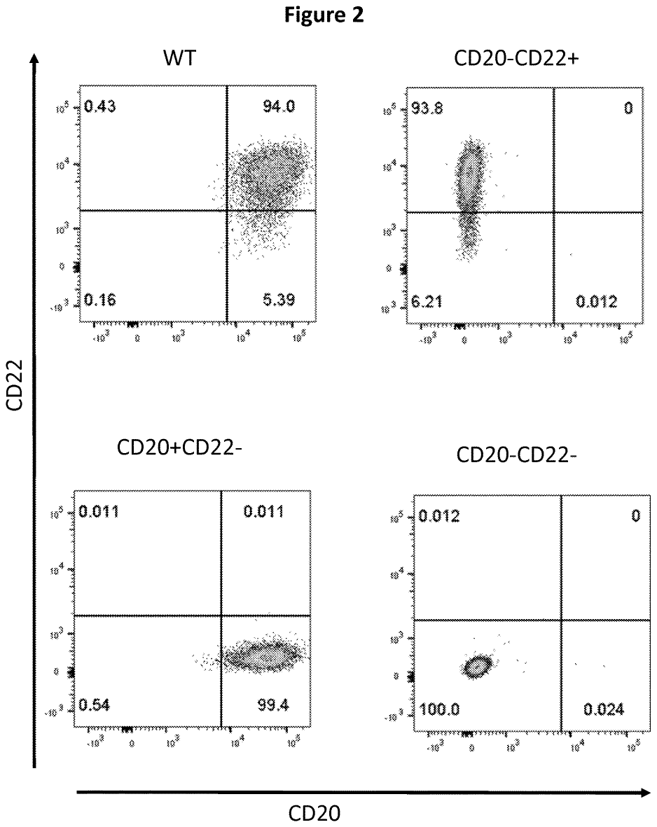

The Raji cell line expresses high level of CD20 and CD22. This cell line was modified to express Luciferase gene and was used as a positive control i.e. for target cells expressing both CD20 and CD22. In addition, Raji cell lines were treated with CD22 and/or CD20 TALEN. Briefly, Raji cells were electroporated with mRNAs encoding CD22 TALEN arms (SEQ ID NO: 27 and SEQ ID NO: 28) and mRNAs encoding CD20 TALEN (SEQ ID NO: 29 and SEQ ID NO: 30) using AgilePulse Max system. Cells were incubated at 30° C. for 16 to 18 hours in RPMI1640 10% Foetal Bovine Serum (FBS) and 1% Penicillin/Streptavidin (culture medium), and were grown in fresh culture medium at 37° C. in % CO2 incubator until sorting. Different cell populations were purified using anti-biotin microbeads and either anti-CD20-biotin, anti-CD22 biotin or both (Biolegend) to obtain distinct cell populations expressing i) CD20 but not CD22, ii) CD22 but not CD20, or iii) not expressing neither CD20 nor CD22. FIG. 2 illustrates the phenotype of these selected populations.

### Example 5. Cytotoxicity of CD20 and CD22 Dual CAR T Cells

CD20xCD22, CD22xCD20 and CD22 CAR T cells as produced in example 1 and coming from three different donors were tested for their cytotoxicity capacity against the different Raji cells generated in example 4.

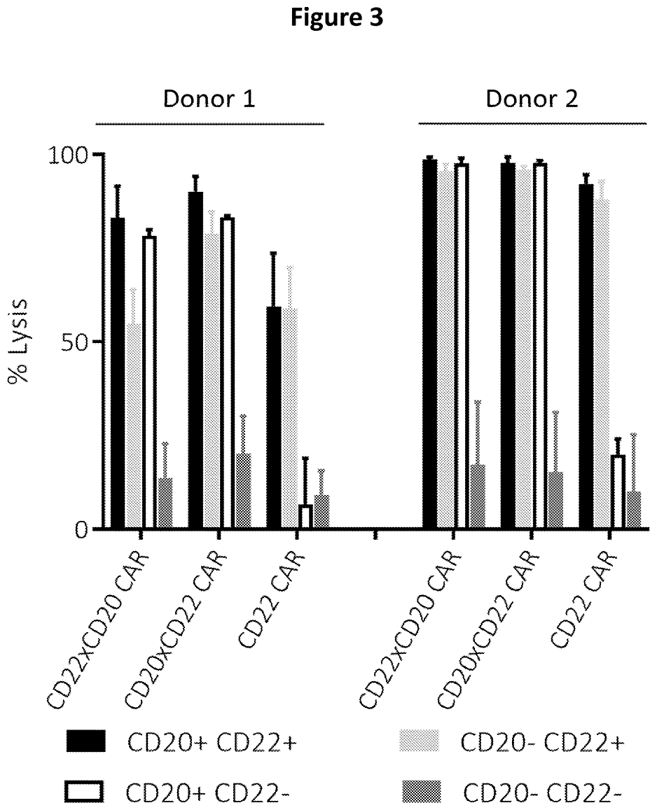

The T cells were thawed and viable cells were counted using NucleoCounter® device (Chemometec NC-250). The different CAR T cells were co-cultured for 4 to 16 hours (at 37° C. in a 5% CO₂ incubator) with the different Raji cells (CD20+CD22+, CD20+CD22−, CD22−CD20+ and CD20−CD22−) expressing luciferase in 96 well plates at different Effector/Target ratio. At the end of the incubation period, the released luciferase was measured using One-Glo® kit (Promega #E6110) according to provider's protocol. FIG. 3 shows that for all donors used, CD22 CAR T cells could efficiently lyse CD20+CD22+ and CD20-CD22+ Raji clones while CD22xCD20 or CD20xCD22 CAR T cells add the same level of lysis than CD22 CAR but could also lyse to the same level CD20+CD22− Raji clones.

The killing ability of CD20xCD22, CD22xCD20 and CD22 CAR T cells as produced in example 1 was also tested in a rechallenge or serial killing assay. The different CAR T cells were thawed, counted and incubated with the different Raji cells at a 1:1 ratio of CAR positive T cells/Raji cells (CD20+CD22+, CD20−CD22+, CD20+CD22−). After 3 days of incubation, half of the wells were used to measure the release luciferase using One-Glo® kit (Promega #E6110), while the other half is transferred to new plates containing Raji cells for an additional incubation. Luciferase measurement was repeated at 7 days, 10 days, 14 days and 17 days.

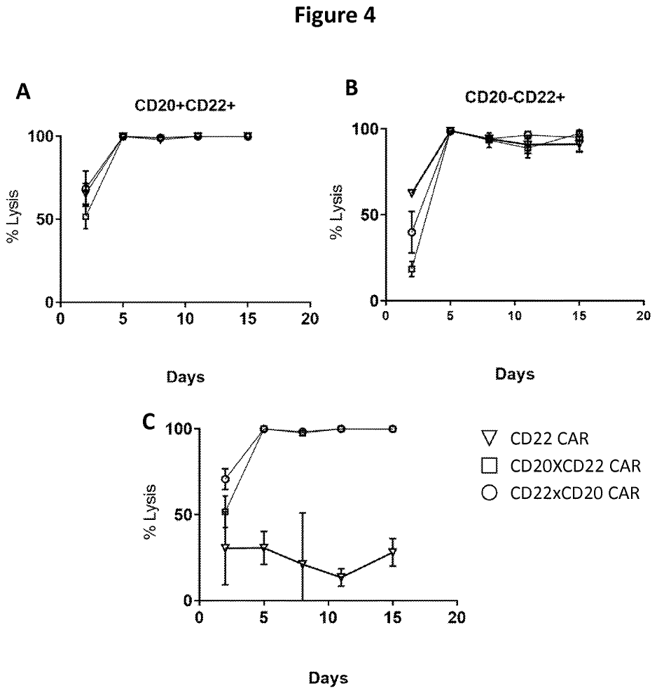

FIG. 4 shows that CD22xCD20 or CD20xCD22 CAR T cells were able to kill efficiently all Raji cells tested whereas CD22 CAR T cells were able to kill efficiently only CD20+CD22+ and CD20−CD22+ Raji cells. Surprisingly, CD22 CAR T had a low activity on CD20+CD22− Raji cells suggesting that CD22 expression was not totally abolished in this clone. This also suggests that dual CARs could efficiently kill targeted tumor cells that have low expression of both targeted antigens.

TABLE 7
List of TALENs used

| Target | Target sequence | TALE-nuclease arms |
|---|---|---|
| TRAC_T01 | TTGTCCCACAGATATCC | TRAC_T01-L TALEN |
| | agaaccctgaccctg | (SEQ ID NO: 23) |
| | CCGTGTACCAGCTGAGA | TRAC_T01-R TALEN |
| | (SEQ ID NO: 29) | (SEQ ID NO: 24) |
| CD52_T01 | TTCCTCCTACTCACCAT | CD52_T01-L TALEN |
| | cagcctcctggttat | (SEQ ID NO: 25) |
| | GGTACAGGTAAGAGCAA | CD52_T01-R TALEN |
| | (SEQ ID NO: 30) | (SEQ ID NO: 26) |
| CD22_T01 | TCTGGTTTTCTTCCAGA | CD22_T01-L TALEN |
| | tcctcccaagaaggt | (SEQ ID NO: 27) |
| | GACCACAGTGATTCAAA | CD22_T01-R TALEN |
| | (SEQ ID NO: 35) | (SEQ ID NO: 28) |
| CD20_T01 | TTTGCTGCCATTTCTGG | CD20_T01-L TALEN |
| | aatgattctttcaat | (SEQ ID NO: 29) |
| | CATGGACATACTTAATA | CD20_T01-R TALEN |
| | (SEQ ID NO: 36) | (SEQ ID NO: 30) |

### Example 6. Tumor Burden Control of a Disseminated In Vivo Model of B-Cell Lymphoma

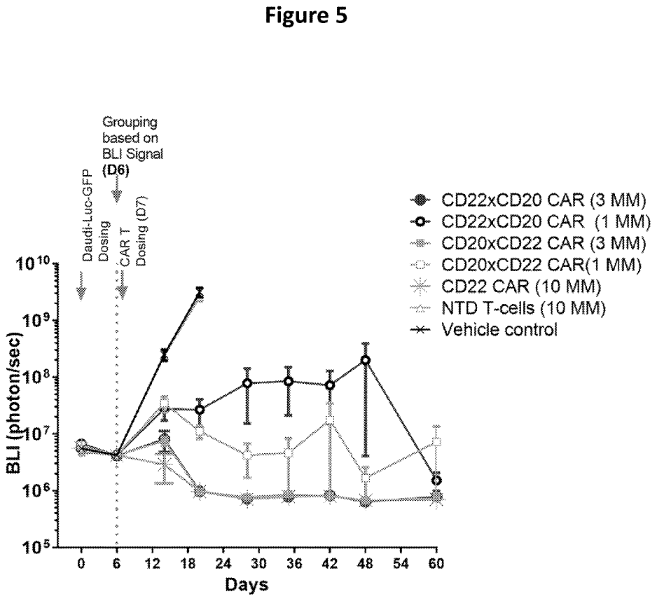

Daudi cells expressing CD20 and CD22 were modified to express luciferase and GFP. Daudi cells were intravenously injected in NSG immunodeficient mice (NOD.Cg-Prkdcscid Il2rgtm1Wjl/SzJ, Jackson laboratories). Seven days after tumor implantation, CD20xCD22 (1 and 3 millions), CD22xCD20 (1 and 3 millions) and CD22 (10 millions) CAR T-cells were intravenously injected in individual mice. Bioluminescence signal (BLI) upon injection of D-luciferin was monitored bi-weekly until day 60 after CAR T-cell injection and values are represented in FIG. 5. For all conditions of treatment with CART-cells tested, BLI signal was reduced compared to vehicle control or non-transduced T cells. Surprisingly, at a low dose of 1 million CAR+ T-cells, CD20xCD22 CAR T-cells had a strong killing activity that was higher than that of CD22xCD20 CAR T-cells.

### Example 7. Treatment Efficacy in a Disseminated In Vivo Model of B-Cell Lymphoma

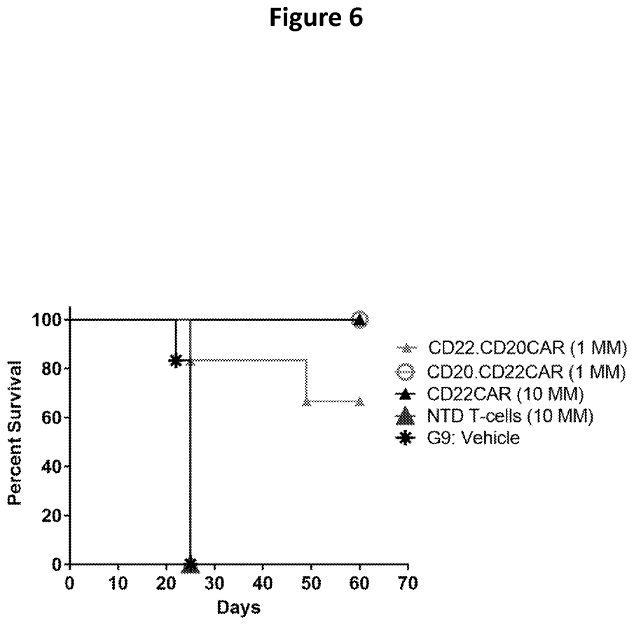

Animals injected with Daudi cells and subsequently intravenously injected with CD20xCD22 (1 million), CD22xCD20 (1 million) and CD22 (10 millions) CAR T-cells from Example 6 were monitored for survival during 60 days. As FIG. 6 shows, animals treated with any of the CART-cells tested survived longer than animals treated with either vehicle or non-transduced T cells (NTD). In addition, and surprisingly, animals treated with CD20xCD22 CAR T-cells survived longer than animals treated with CD22xCD20 CAR T-cells.

### Example 8. Treatment Efficacy in a Disseminated In Vivo Model of B-Cell Lymphoma

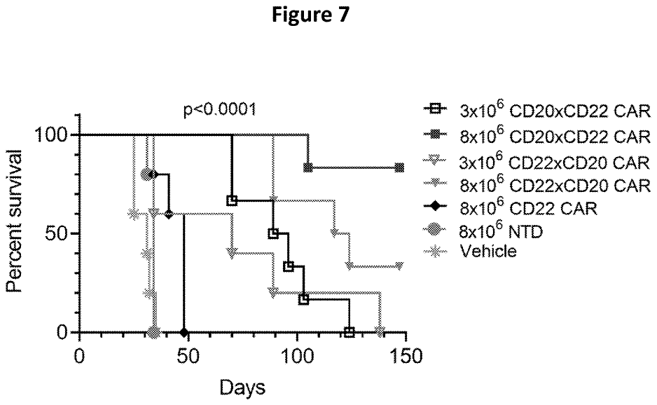

Raji cell lines described in Example 2 were injected subcutaneously in NSG immunodeficient mice (NOD.Cg-Prkdcscid Il2rgtm1Wjl/SzJ, Jackson laboratories). All three cell lines were injected simultaneously in each individual animal, specifically, Raji WT cells were injected in one flank, Raji CD22− cells were injected in another flank and CD20− cells in a third flank. One week after tumor injection, CD20xCD22 (3 and 8 millions), CD22xCD20 (3 and 8 millions) and CD22 (8 millions) CAR T-cells were injected intravenously and animals were monitored for survival. As depicted in FIG. 7, animals treated with CD22 CAR T-cells succumbed fast to the disease since they carried CD22− tumor cells, which can not be targeted by CD22 CAR T-cells. Treatment with three and eight millions of dual CAR T-cells shows that while both treatments are efficient, CD20xCD22 treated animals surprisingly survived for a longer time than animals treated with CD22xCD20.

### Example 9. IFN Gamma Release of CD20 and CD22 Dual CAR T Cells

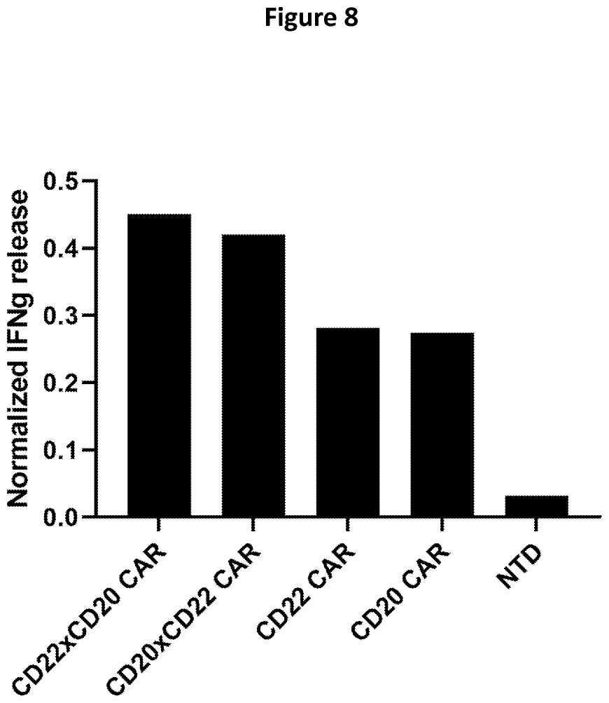

Daudi cells expressing CD20 and CD22 were modified to express luciferase gene and GFP. Those Daudi cells were incubated overnight with CD20xCD22, CD22xCD20, CD22 or CD20 positive CAR T-cells at a ratio of 1:1 (effector-target). Next day, plate was centrifuged, and supernatant was collected. Levels of IFN gamma released upon incubation of tumor with CAR T-cells were quantified using the Human IFN-gamma Quantikine ELISA kit (R&D systems, DIF50C) following manufacturer's instructions. As positive control CAR-T-cells were incubated with phorbol myristate acetate (PMA, Sigma Aldrich P8139) and lonomycin (Sigma aldrich 10634). Final values of IFN gamma release were normalized to those of the positive control. FIG. 8 shows that dual CD20xCD22 CAR-T cells as well as CD22xCD20 CAR T cells were activated upon antigen recognition on tumor cells and released higher levels of IFN-gamma than the single CAR (CD20 or CD22) T cells. This observation points towards a synergistic benefit of using the dual CD20, CD22 CAR T cells.

1. A genetically engineered immune cell expressing a Chimeric Antigen Receptor (CAR) specific for CD22 (CAR22) and a CAR specific for CD20 (CAR20) at its cell surface, wherein said CAR22 and said CAR20 are encoded by an exogenous nucleic acid incorporated in the genome of said immune cell, and wherein said exogenous nucleic acid comprises, from 5′ to 3′:
a) a promoter,
b) a nucleic acid encoding said CAR20,
c) a nucleic acid encoding a self-cleaving peptide, and
d) a nucleic acid encoding said CAR22,
wherein the promoter of a) controls expression of said CAR20 and said CAR22.

2. The genetically engineered immune cell of claim 1,
a) wherein said CAR22 comprises:
i) at least one extracellular domain comprising:
an antigen binding domain specific for CD22 comprising a Variable Heavy chain (VH) comprising an amino acid sequence having at least 80% identity with SEQ ID NO: 11 and comprising the H-CDRs of amino acid sequences SEQ ID NO: 41, SEQ ID NO: 42, and SEQ ID NO: 43, and a Variable Light chain (VL) comprising an amino acid sequence having at least 80% identity with SEQ ID NO: 12 and comprising the L-CDRs of amino acid sequences SEQ ID NO: 44, SEQ ID NO: 45, and SEQ ID NO: 46,
a hinge domain from CD8alpha,
ii) a transmembrane domain from CD8alpha, and
iii) an intracellular domain comprising a 4-1BB stimulatory domain and a CD3zeta signaling domain; and
b) wherein said CAR20 comprises:
i) at least one extracellular domain comprising:
an antigen binding domain specific for CD20 comprising a Variable Heavy chain (VH) comprising an amino acid sequence having at least 80% identity with SEQ ID NO: 15 and comprising the H-CDRs of amino acid sequences SEQ ID NO: 47, SEQ ID NO: 48, and SEQ ID NO: 49, and a Variable Light chain (VL) comprising an amino acid sequence having at least 80% identity with SEQ ID NO: 16 and comprising the L-CDRs of amino acid sequences SEQ ID NO: 50, SEQ ID NO: 51, and SEQ ID NO: 52,
a hinge domain from CD8alpha,
ii) a transmembrane domain from CD8alpha, and
iii) an intracellular domain comprising a 4-1 BB stimulatory domain and a CD3zeta signaling domain.

3. The genetically engineered immune cell of claim 1, wherein said engineered immune cell is selected from the group consisting of a T-cell, a NK-cell, and a macrophage.

4. The genetically engineered immune cell of claim 1, wherein said engineered immune cell is a T-cell.

5. The genetically engineered immune cell of claim 4, wherein said T-cell is a cytotoxic T cell.

6. The genetically engineered immune cell of claim 1, wherein said engineered immune cell is a T-cell that has at least one allele encoding TCR alpha, TCR beta, and/or CD3 that has been inactivated by mutation.

7. The genetically engineered immune cell of claim 1, wherein said engineered immune cell is a T-cell that has at least one allele selected from β2m, PD1, CTLA4, dCK, CD52 and GR that has been inactivated.

8. The genetically engineered immune cell of claim 1, wherein said engineered immune cell is a cytotoxic T-cell that has at least one allele encoding TCR alpha, TCR beta, CD3, and/or CD52 that has been inactivated.

9. A pharmaceutical composition comprising the genetically engineered immune cell of claim 1 or a population of cells comprising the genetically engineered immune cell of claim 1, and a pharmaceutically acceptable excipient.

10. An isolated polynucleotide comprising, from 5′ to 3′:
a) a promoter (such as the EF1alpha promoter);
b) a nucleic acid encoding a CAR20, wherein said CAR20 comprises:
i) at least one extracellular domain comprising:
an antigen binding domain specific for CD20 comprising a Variable Heavy chain (VH) comprising an amino acid sequence having at least 80%, at least 90%, at least 95%, at least 97%, at least 99%, or 100% identity with SEQ ID NO: 15 and comprising the H-CDRs of amino acid sequences SEQ ID NO: 47, SEQ ID NO: 48, and SEQ ID NO: 49, and a Variable Light chain (VL) comprising an amino acid sequence having at least 80%, at least 90%, at least 95%, at least 97%, at least 99%, or 100% identity with SEQ ID NO: 16 and comprising the L-CDRs of amino acid sequences SEQ ID NO: 50, SEQ ID NO: 51, and SEQ ID NO: 52, optionally a leader sequence,
a hinge domain from CD8alpha,
ii) a transmembrane domain from CD8alpha, and
iii) an intracellular domain comprising a 4-1BB stimulatory domain and a CD3zeta signaling domain;
c) a nucleic acid sequence encoding a self-cleaving peptide (such as P2A, T2A, E2A, or F2A);
d) a nucleic acid encoding a CAR22, wherein said CAR22 comprises:
i) at least one extracellular domain comprising:
an antigen binding domain specific for CD22 comprising a Variable Heavy chain (VH) comprising an amino acid sequence having at least 80%, at least 90%, at least 95%, at least 97%, at least 99%, or 100% identity with SEQ ID NO: 11 and comprising the H-CDRs of amino acid sequences SEQ ID NO: 41, SEQ ID NO: 42, and SEQ ID NO: 43, and a Variable Light chain (VL) comprising an amino acid sequence having at least 80%, at least 90%, at least 95%, at least 97%, at least 99%, or 100% identity with SEQ ID NO: 12 and comprising the L-CDRs of amino acid sequences SEQ ID NO: 44, SEQ ID NO: 45, and SEQ ID NO: 46, optionally a leader sequence,
a hinge domain from CD8alpha,
ii) a transmembrane domain from CD8alpha, and
iii) an intracellular domain comprising a 4-1BB stimulatory domain and a CD3zeta signaling domain;
wherein the promoter of a) controls expression of said CAR20 and said CAR22.

11. A method of preparing a genetically engineered immune cell expressing a Chimeric Antigen Receptor (CAR) specific for CD22 (CAR22) and a CAR specific for CD20 (CAR20) at its cell surface, wherein said CAR22 and said CAR20 are encoded by an exogenous nucleic acid incorporated in the genome of said genetically engineered immune cell, and wherein said exogenous nucleic acid comprises, from 5′ to 3′:
a) a promoter,
b) a nucleic acid encoding said CAR20,
c) a nucleic acid encoding a self-cleaving peptide, and
d) a nucleic acid encoding said CAR22,
wherein the promoter of a) controls expression of said CAR20 and said CAR22, and wherein said method comprises introducing into an immune cell the isolated polynucleotide of claim 10, or a vector comprising said polynucleotide.

12. A method of treatment of a patient suffering from a cancer associated with CD20 and/or CD22 expression, said method comprising administering to said patient an effective amount of a genetically engineered immune cell of claim 1.

13. The method of treatment of claim 12, wherein said cancer is selected from the group consisting of lymphoma, Hodgkin lymphoma (HL), non-Hodgkin lymphoma (NHL), leukemia, multiple myeloma (MM), B-chronic lymphocytic leukemia (B-CLL), hairy cell leukemia (HCL), acute lymphocytic leukemia (ALL), acute lymphocytic cancer, and acute myeloid leukemia (AML).

14. A genetically engineered T cell expressing a Chimeric Antigen Receptor (CAR) that specifically binds to CD20 (CAR20) and a CAR that specifically binds to CD22 (CAR22) at its cell surface, wherein said CAR22 and said CAR20 are encoded by an exogenous nucleic acid incorporated in the genome of said T cell, and wherein said exogenous nucleic acid comprises, from 5′ to 3′:
a) a promoter,
b) a nucleic acid encoding said CAR20, wherein said CAR20 comprises the amino acid sequence of SEQ ID NO: 18, optionally without the leader sequence of SEQ ID NO: 1,
c) a nucleic acid encoding a self-cleaving peptide, and
d) a nucleic acid encoding said CAR22, wherein said CAR22 comprises the amino acid sequence of SEQ ID NO: 14, optionally without the leader sequence of SEQ ID NO: 1,
wherein the promoter of a) controls expression of said CAR20 and said CAR22;
wherein said genetically engineered T cell comprises at least one allele encoding TCR alpha, TCR beta, and/or CD3 that has been inactivated; and
wherein said genetically engineered T cell has at least one inactivated CD52 allele.

15. A population of immune cells comprising the genetically engineered T cell of claim 14.

16. A pharmaceutical composition comprising a pharmaceutically acceptable excipient and (a) the genetically engineered T cell of claim 14 or (b) a population of immune cells comprising the genetically engineered T cell of claim 14.

17. A method of treating a patient having a cancer associated with CD20 expression, CD22 expression, or a combination of CD20 expression and CD22 expression, wherein said method comprises administering to said patient an effective amount of the genetically engineered T cell of claim 14.

18. The method of claim 17, wherein said cancer is selected from the group consisting of lymphoma, Hodgkin lymphoma (HL), non-Hodgkin lymphoma (NHL), leukemia, multiple myeloma (MM), B-chronic lymphocytic leukemia (B-CLL), hairy cell leukemia (HCL), acute lymphocytic leukemia (ALL), acute lymphocytic cancer, and acute myeloid leukemia (AML).


SEQUENCE LISTING (52 sequences)

SEQ ID NO: 1 - protein, 21 aa, synthetic construct
MALPVTALLLPLALLLHAARP

SEQ ID NO: 2 - protein, 20 aa, synthetic construct
METDTLLLWVLLLWVPGSTG

SEQ ID NO: 3 - protein, 16 aa, synthetic construct
GLAVSTISSFFPPGYQ

SEQ ID NO: 4 - protein, 45 aa, synthetic construct
TTTPAPRPPTPAPTIASQPLSLRPEACRPAAGGAVHTRGLDFACD

SEQ ID NO: 5 - protein, 231 aa, synthetic construct
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 24 aa, synthetic construct
IYIWAPLAGTCGVLLLSLVITLYC

SEQ ID NO: 7 - protein, 27 aa, synthetic construct
IISFFLALTSTALLFLLFFLTLRFSVV

SEQ ID NO: 8 - protein, 42 aa, synthetic construct
KRGRKKLLYIFKQPFMRPVQTTQEEDGCSCRFPEEEEGGCEL

SEQ ID NO: 9 - protein, 112 aa, synthetic construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 10 - protein, 15 aa, synthetic construct
GGGGSGGGGSGGGGS

SEQ ID NO: 11 - protein, 124 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSS

SEQ ID NO: 12 - protein, 107 aa, synthetic construct
DIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 13 - protein, 246 aa, synthetic construct
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIK

SEQ ID NO: 14 - protein, 490 aa, synthetic construct
MALPVTALLLPLALLLHAARPQVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCAREVTGDLEDAFDIWGQGTMVTVSSGGGGSGGGGSGGGGSDIQMTQSPSSLSASVGDRVTITCRASQTIWSYLNWYQQRPGKAPNLLIYAASSLQSGVPSRFSGRGSGTDFTLTISSLQAEDFATYYCQQSYSIPQTFGQGTKLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - protein, 122 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 16 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 17 - protein, 244 aa, synthetic construct
EVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIK

SEQ ID NO: 18 - protein, 488 aa, synthetic construct
MALPVTALLLPLALLLHAARPEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - protein, 22 aa, synthetic construct
GSGATNFSLLKQAGDVEENPGP

SEQ ID NO: 20 - protein, 317 aa, synthetic construct
MTTPRNSVNGTFPAEPMKGPIAMQSGPKPLFRRMSSLVGPTQSFFMRESKTLGAVQIMNGLFHIALGGLLMIPAGIYAPICVTVWYPLWGGIMYIISGSLLAATEKNSRKCLVKGKMIMNSLSLFAAISGMILSIMDILNIKISHFLKMESLNFIRAHTPYINIYNCEPANPSEKNSPSTQYCYSIQSLFLGILSVMLIFAFFQELVIAGIVENEWKRTCSRPKSNIVLLSAEEKKEQTIEIKEEVVGLTETSSQPKNEEDIEIIPIQEEEEEETETNFPEPPQDQESSPIENDSSPGGGSGGGSHHHHHHHHHHHH

SEQ ID NO: 21 - protein, 916 aa, synthetic construct
MEWSWVFLFFLSVTTGVHSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRGGGGAGGGGCKPCICTVPEVSSVFIFPPKPKDVLTITLTPKVTCVVVDISKDDPEVQFSWFVDDVEVHTAQTQPREEQFNSTFRSVSELPIMHQDWLNGKEFKCRVNSAAFPAPIEKTISKTKGRPKAPQVYTIPPPKEQMADKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 22 - protein, 9 aa, synthetic construct
CPYSNPSLC

SEQ ID NO: 23 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 24 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 25 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 26 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 27 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 28 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 29 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 30 - protein, 925 aa, synthetic construct
MGDPKKKRKVIDIADLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLNLTPEQVVAIASNIGGKQALETVQALLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPQQVVAIASNGGGRPALESIVAQLSRPDPALAALTNDHLVALACLGGRPALDAVKKGLGDPISRSQLVKSELEEKKSELRHKLKYVPHEYIELIEIARNSTQDRILEMKVMEFFMKVYGYRGKHLGGSRKPDGAIYTVGSPIDYGVIVDTKAYSGGYNLPIGQADEMQRYVEENQTRNKHINPNEWWKVYPSSVTEFKFLFVSGHFKGNYKAQLTRLNHITNCNGAVLSVEELLIGGEMIKAGTLTLEEVRRKFNNGEINFAAD

SEQ ID NO: 31 - DNA, 3003 bp, synthetic construct
  source 1..3003: mol_type = other DNA
atggctctgcccgtcaccgctctgctgctgccactggccctgctgctgcacgcagcaagaccacaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgtctagcggaggaggaggatccggaggaggaggatctggcggcggcggcagcgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctgcaggccgaggacttcgccacctactattgccagcagtcttatagcatcccccagacatttggccagggcaccaagctggagatcaagaccacaaccccagcaccaaggccacctacacctgcaccaaccatcgcctctcagcccctgagcctgagacctgaggcatgtaggccagcagcaggaggagcagtccatacaaggggtctggattttgcatgcgacatctacatctgggcacctctggcaggaacatgtggcgtgctcctgctcagcctggtcatcaccctgtactgcaagagaggcaggaagaagctgctgtatatcttcaagcagcccttcatgcgccccgtgcagacaacccaggaggaggatggctgctcctgtaggttcccagaagaggaggagggaggatgtgagctgcgcgtgaagttttcccggtctgccgacgcacctgcataccagcagggccagaaccagctgtataacgagctgaatctgggccggagagaggagtacgatgtgctggacaagaggcgcggcagagatccagagatgggcggcaagccccggagaaagaaccctcaggagggcctgtacaatgagctgcagaaggataagatggccgaggcctattctgagatcggcatgaagggagagaggcgccggggcaagggacacgacggactgtaccagggactgagcacagccaccaaggatacctatgacgccctgcatatgcaggcactgcctccaaggggaagcggagctactaacttcagcctgctgaagcaggctggagacgtggaggagaaccctggacctatggctctgcctgtcaccgctctgctgctgcccctggctctgctgctgcacgccgcacgccccgaagtccagctggtcgaatctgggggcgggctggtgcagccaggaagatcactgaggctgagctgcgccgcttccggcttcaccttcaacgactatgccatgcactgggtgagacaggctcccggaaagggcctggagtgggtctctaccatcagttggaattccgggtctattggatatgccgacagcgtgaaaggccgcttcacaatctctcgagataacgctaagaaaagtctgtacctgcagatgaattcactgagggcagaggacactgccctgtactattgcgccaaggatattcagtacggcaactactattacgggatggacgtctgggggcagggaaccacagtgaccgtcagctccggaggaggaggatccggaggaggaggaagcggaggaggaggatccgagatcgtgctgacacagagcccagccactctgagtctgtcacccggcgaacgagctacactgtcctgtcgggcaagccagtccgtctctagttatctggcttggtaccagcagaagccaggacaggcaccacgactgctgatctacgatgctagcaacagagcaacagggattcctgcaaggttctctggcagtgggtcaggaactgactttacactgactatctcaagcctggagcctgaagatttcgccgtgtattactgccagcagcggtccaattggccaatcacctttggccaggggacacgcctggagatcaagactaccacacctgctccacgaccacctactccagcacctaccattgcttctcagcccctgagtctgcggcctgaagcatgtagaccagcagcaggaggagcagtgcataccagaggactggactttgcctgcgatatctatatttgggcaccactggctggaacttgtggagtgctgctgctgtctctggtcatcaccctgtattgcaagcgaggccggaagaaactgctgtacattttcaaacagccttttatgagaccagtgcagactacccaggaggaagacggctgcagctgtaggttccccgaggaagaggaagggggatgtgagctgagggtcaagtttagccgctccgctgatgcacctgcctatcagcaggggcagaatcagctgtacaacgagctgaatctgggacggagagaggaatacgacgtgctggataaaaggcgaggacgagatccagaaatgggagggaagccccgacggaaaaaccctcaggagggcctgtataatgaactgcagaaggacaaaatggctgaggcatactctgaaatcggaatgaagggcgagagaaggcgcggaaaaggccacgatgggctgtatcagggactgagtaccgccacaaaggacacctacgatgcactgcatatgcaggccctgccaccccggtga

SEQ ID NO: 32 - DNA, 3003 bp, synthetic construct
  source 1..3003: mol_type = other DNA
atggctctgcctgtcaccgctctgctgctgcccctggctctgctgctgcacgccgcacgccccgaagtccagctggtcgaatctgggggcgggctggtgcagccaggaagatcactgaggctgagctgcgccgcttccggcttcaccttcaacgactatgccatgcactgggtgagacaggctcccggaaagggcctggagtgggtctctaccatcagttggaattccgggtctattggatatgccgacagcgtgaaaggccgcttcacaatctctcgagataacgctaagaaaagtctgtacctgcagatgaattcactgagggcagaggacactgccctgtactattgcgccaaggatattcagtacggcaactactattacgggatggacgtctgggggcagggaaccacagtgaccgtcagctccggaggaggaggatccggaggaggaggaagcggaggaggaggatccgagatcgtgctgacacagagcccagccactctgagtctgtcacccggcgaacgagctacactgtcctgtcgggcaagccagtccgtctctagttatctggcttggtaccagcagaagccaggacaggcaccacgactgctgatctacgatgctagcaacagagcaacagggattcctgcaaggttctctggcagtgggtcaggaactgactttacactgactatctcaagcctggagcctgaagatttcgccgtgtattactgccagcagcggtccaattggccaatcacctttggccaggggacacgcctggagatcaagactaccacacctgctccacgaccacctactccagcacctaccattgcttctcagcccctgagtctgcggcctgaagcatgtagaccagcagcaggaggagcagtgcataccagaggactggactttgcctgcgatatctatatttgggcaccactggctggaacttgtggagtgctgctgctgtctctggtcatcaccctgtattgcaagcgaggccggaagaaactgctgtacattttcaaacagccttttatgagaccagtgcagactacccaggaggaagacggctgcagctgtaggttccccgaggaagaggaagggggatgtgagctgagggtcaagtttagccgctccgctgatgcacctgcctatcagcaggggcagaatcagctgtacaacgagctgaatctgggacggagagaggaatacgacgtgctggataaaaggcgaggacgagatccagaaatgggagggaagccccgacggaaaaaccctcaggagggcctgtataatgaactgcagaaggacaaaatggctgaggcatactctgaaatcggaatgaagggcgagagaaggcgcggaaaaggccacgatgggctgtatcagggactgagtaccgccacaaaggacacctacgatgcactgcatatgcaggccctgccaccccgaggaagcggagctactaacttcagcctgctgaagcaggctggagacgtggaggagaaccctggacctatggctctgcccgtcaccgctctgctgctgccactggccctgctgctgcacgcagcaagaccacaggtgcagctgcagcagagcggccctggcctggtgaagccaagccagacactgtccctgacctgcgccatcagcggcgattccgtgagctccaactccgccgcctggaattggatcaggcagtccccttctcggggcctggagtggctgggaaggacatactatcggtctaagtggtacaacgattatgccgtgtctgtgaagagcagaatcacaatcaaccctgacacctccaagaatcagttctctctgcagctgaatagcgtgacaccagaggacaccgccgtgtactattgcgccagggaggtgaccggcgacctggaggatgcctttgacatctggggccagggcacaatggtgaccgtgtctagcggaggaggaggatccggaggaggaggatctggcggcggcggcagcgatatccagatgacacagtccccatcctctctgagcgcctccgtgggcgacagagtgacaatcacctgtagggcctcccagaccatctggtcttacctgaactggtatcagcagaggcccggcaaggcccctaatctgctgatctacgcagcaagctccctgcagagcggagtgccatccagattctctggcaggggctccggcacagacttcaccctgaccatctctagcctgcaggccgaggacttcgccacctactattgccagcagtcttatagcatcccccagacatttggccagggcaccaagctggagatcaagaccacaaccccagcaccaaggccacctacacctgcaccaaccatcgcctctcagcccctgagcctgagacctgaggcatgtaggccagcagcaggaggagcagtccatacaaggggtctggattttgcatgcgacatctacatctgggcacctctggcaggaacatgtggcgtgctcctgctcagcctggtcatcaccctgtactgcaagagaggcaggaagaagctgctgtatatcttcaagcagcccttcatgcgccccgtgcagacaacccaggaggaggatggctgctcctgtaggttcccagaagaggaggagggaggatgtgagctgcgcgtgaagttttcccggtctgccgacgcacctgcataccagcagggccagaaccagctgtataacgagctgaatctgggccggagagaggagtacgatgtgctggacaagaggcgcggcagagatccagagatgggcggcaagccccggagaaagaaccctcaggagggcctgtacaatgagctgcagaaggataagatggccgaggcctattctgagatcggcatgaagggagagaggcgccggggcaagggacacgacggactgtaccagggactgagcacagccaccaaggatacctatgacgccctgcatatgcaggcactgcctccaaggtga

SEQ ID NO: 33 - DNA, 49 bp, synthetic construct
  source 1..49: mol_type = other DNA
ttgtcccacagatatccagaaccctgaccctgccgtgtaccagctgaga

SEQ ID NO: 34 - DNA, 49 bp, synthetic construct
  source 1..49: mol_type = other DNA
ttcctcctactcaccatcagcctcctggttatggtacaggtaagagcaa

SEQ ID NO: 35 - DNA, 49 bp, synthetic construct
  source 1..49: mol_type = other DNA
tctggttttcttccagatcctcccaagaaggtgaccacagtgattcaaa

SEQ ID NO: 36 - DNA, 49 bp, synthetic construct
  source 1..49: mol_type = other DNA
tttgctgccatttctggaatgattctttcaatcatggacatacttaata

SEQ ID NO: 37 - DNA, 588 bp, synthetic construct
  source 1..588: mol_type = other DNA
gcccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataatatggccacaacc

SEQ ID NO: 38 - protein, 21 aa, synthetic construct
GSGEGRGSLLTCGDVEENPGP

SEQ ID NO: 39 - protein, 23 aa, synthetic construct
GSGQCTNYALLKLAGDVESNPGP

SEQ ID NO: 40 - protein, 25 aa, synthetic construct
GSGVKQTLNFDLLKLAGDVESNPGP

SEQ ID NO: 41 - protein, 11 aa, synthetic construct
SGDSVSSNSAA

SEQ ID NO: 42 - protein, 9 aa, synthetic construct
TYYRSKWYN

SEQ ID NO: 43 - protein, 13 aa, synthetic construct
AREVTGDLEDAFD

SEQ ID NO: 44 - protein, 6 aa, synthetic construct
QTIWSY

SEQ ID NO: 45 - skipped

SEQ ID NO: 46 - protein, 9 aa, synthetic construct
QQSYSIPQT

SEQ ID NO: 47 - protein, 8 aa, synthetic construct
GFTFNDYA

SEQ ID NO: 48 - protein, 8 aa, synthetic construct
ISWNSGSI

SEQ ID NO: 49 - protein, 15 aa, synthetic construct
AKDIQYGNYYYGMDV

SEQ ID NO: 50 - protein, 6 aa, synthetic construct
QSVSSY

SEQ ID NO: 51 - skipped

SEQ ID NO: 52 - protein, 9 aa, synthetic construct
QQRSNWPIT